(19) United States
(12) Patent Application Publication
(10) Pub. No.: US 2014/0023656 A1
(43) Pub. Date: Jan. 23, 2014

(54) PHARMACEUTICAL COMPOSITION FOR TREATMENT AND PREVENTION OF TH3 MARCH-RELATED DISEASES

(71) Applicants: Masato Kuwabara, Kanagawa (JP); Yasuhiko Kojima, Kanagawa (JP); Takashi Oyamada, Gunma (JP); Michio Honjo, Tokyo (JP); Kazuko Uno, Osaka (JP); Yukio Yajima, Saitama (JP); Akina Kuwabara, Kanagawa (JP)

(72) Inventors: Masato Kuwabara, Kanagawa (JP); Yasuhiko Kojima, Kanagawa (JP); Takashi Oyamada, Gunma (JP); Michio Honjo, Tokyo (JP); Kazuko Uno, Osaka (JP); Yukio Yajima, Saitama (JP); Akina Kuwabara, Kanagawa (JP)

(73) Assignee: BioLT CO., LTD., Akiruno-shi, Tokyo (JP)

(21) Appl. No.: 13/824,672

(22) PCT Filed: Sep. 26, 2011
(86) PCT No.: PCT/JP2011/072700

§ 371 Date: May 31, 2013

(30) Foreign Application Priority Data
Sep. 24, 2010 (JP) 2010-214531

(51) Int. Cl. A61K 33/30; G01N 33/68

(52) U.S. Cl. CPC A61K 33/30; G01N 33/6893
USPC 424/158.1; 424/641; 435/7.92

(57) ABSTRACT

The present invention provides a medicinal agent and a method for each of various testing or the like, which enable the treatment and prevention, particularly preventive treatment, of Th3 march-related diseases. The present invention also provides a pharmaceutical composition for treating and/or preventing Th3 march-related diseases, which comprises zinc, calcium and phosphorus and additionally comprises pumpkin seeds and corn silk, and which preferably can activate the DNA repairing ability of a zinc finger, particularly the ability of repairing defect or mutation of DNA for a filaggrin gene, of a zinc finger.

### TECHNICAL FIELD

The present invention relates to a pharmaceutical composition for treatment and prevention of Th3 march-related diseases, a method for treatment, prevention and diagnosis of Th3 march-related diseases, a method for testing classification of treatment and prevention, a method for detecting intention to use a therapeutic drug and a prophylactic of Th3 march-related diseases, a kit for testing classification of treatment and prevention of Th3 march-related diseases, a kit for detecting intention to use a therapeutic drug and/or a prophylactic of Th3 march-related diseases, and a diagnosis kit of Th3 march-related diseases.

### BACKGROUND OF THE INVENTION

In recent years, it has been said that since there is a possibility to have different causes and treatment methods in the treatment of immune diseases such as allergic diseases depending upon patients even if a patient has a similar symptom in case of human having various inheritance backgrounds, it is very important to understand immune functions possessed by an individual patient. However, the understanding of the immune functions has only been suggested from the results of animal researches and the like, no concrete method applicable to treatment has yet been clarified.

As one of characteristics of allergic diseases, it has been known that there is so-called allergy march, i.e., linkage phenomenon of allergy in which a main symptom of allergy is changed by the age (Peter I Frank et al., “Long term prognosis in preschool children with wheeze: longitudinal postal questionnaire study 1993-2004”, BMJ, vol. 336, p. 1423-1426, 2008). For example, a person who has predisposition to atopy gets atopic dermatitis at an infant period, gets bronchial asthma at a child period, and has the onset of allergic rhinitis at an adult period. Such continuity of symptom and new onsets of symptom due to the allergy march become social problems and are desired to take a rapid countermeasure.

### SUMMARY OF THE INVENTION

We have standardized a novel Th3 march in mammals. This novel Th3 march passes through signal transduction of zinc cell cytokine. Th3 march regulates potassium and phosphor transmitted to cells by cell surface BMP (Bone Morphogenetic Protein) and TGF-β1 (newly defined as TGF-β1/Th3 march) and by IFN and IL-13(Znf) (see FIG. 2A and FIGS. 2B and 2C). Specifically, the order of pathological changes clinically seen depending upon ages is newly defined. Th3 march marches, from juvenile, Th1 vital reaction dependency (bone disease), Th2 vital reaction dependent atopy (skin disease as abnormality of connective tissue), and Th3 vital reaction (inhibitory factor of serous cure). However, there is no concrete treatment nor prevention method, test method, decision method, nor evaluation method of Th3 march related diseases.

In such situations, it has been desired to develop a medicine and various methods such as a test method which can treat and prevent Th3 march related diseases, particularly early intervention of diseases (Mibyou: previous prevention of diseases considered to be developed in the next stage due to marching and, thus intervention of early medical treatment).

The present invention is accomplished considering the above situations, and a novel medicine has created which acts on a transmission rout of an accession region of Th3-march defined as zinc finger (Znf) (DNA repair capacity). Also, we have developed a novel cytokine panel which carry out the evaluation utilizing IL-13 enhanced by the activation of Th9 cells and Znf. Also, we have found various methods such as method for treating, preventing, diagnosing Th3 march-related diseases, and other diagnosing method which evaluates Th3 march with the cytokine panel, and used the evaluated results as in an index, and has developed kits for using these methods.

Specifically, the present invention relates to a pharmaceutical composition for treatment and prevention of Th3 march-related diseases, a method for treatment, prevention and diagnosis of Th3 march-related diseases, a method for testing intention to use a therapeutic drug and a prophylactic of Th3 march-related diseases, a kit for testing classification of treatment and prevention of Th3 march-related diseases, a kit for testing intention to use a therapeutic drug and/or a prophylactic of Th3 march-related diseases, and a diagnosis kit of Th3 march-related diseases as described below.

(1) A pharmaceutical composition (1) for treatment and/or prevention of Th3 march-related diseases comprising zinc, calcium and phosphor.

For example, the pharmaceutical composition may comprise pumpkin seeds and a corn silk.

As the pharmaceutical composition (1), those which activate DNA repair capacity, typically those which activate DNA repair capacity of zinc finger can be mentioned. Gene DNA which is a target of repair of the DNA repair capacity preferably includes, but not restricted to, filaggrin gene. Specifically, as the pharmaceutical composition (1), those which activate repair capacity of deletion or mutation of filaggrin gene DNA are preferable.

We have found that deletion or mutation of filaggrin gene DNA (decreasing of expression amounts of protein thereby) is greatly related not only to Th2 atopic march (Th2 march, atopic march) patients, but also to Th3 march-related diseases. It has been evidenced from the finding that development of whole of Th3 march-related diseases such as psoriasis which is Th1 disease, and Ehlers-Danlos syndrome (EDS) which is Th2 disease, is related to deletion or mutation of filaggrin gene DNA in addition to atopic dermatitis, which is Th2 disease, as described later on in the working examples, and the finding that the deletion or mutation of filaggrin gene DNA can be repaired by activating DNA repair capacity of zinc finger through the pharmaceutical composition (1), whereby the whole of Th3 march-related diseases can be cured or improved.

In the pharmaceutical composition (1), as Th3 march-related diseases at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3, can be mentioned.

(2) A method for treating and/or preventing Th3 march-related diseases comprises: measuring a concentration of cytokine and/or chemokine in blood of a test animal; and initiating, continuing, interrupting or discontinuing an administration of the pharmaceutical composition according to one of claims 1 to 4 using the measured results as an index.
(3) A method for examining the classification of treatment and/or prevention of Th3 march-related diseases comprising: measuring a concentration of cytokine and/or chemokine in blood of a test animal; and examining the classification of treatment and/or prevention of Th3 march-related diseases using the measured results as an index.
(4) A method for deciding intension to use of treatment and/or prevention drug comprising: measuring a concentration of cytokine and/or chemokine in blood of a test animal; and deciding intension to use of treatment and/or prevention drug using the measured results as an index.
(5) A method for diagnosing Th3 march-related diseases comprising measuring a concentration of cytokine and/or chemokine in blood of a test animal; and diagnosing Th3 march-related diseases using the measured results as an index.

In the methods of (2) to (5), cytokine and chemokine may be at least one member selected from TGF-β1, IL-1β, IL-4, IL-5, IL-6, IL-9, IL-10, IL-13, IFN-γ, RANTES, various FGF (Healing Formula). In the methods of (2) to (5), the method may further comprise measuring a concentration of zinc in blood.

In the methods of (2) to (5), Th3 march-related disease may be, for example, selected from at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3.

In the methods of (2) to (5), test animal may be a human or non-human animal, and a dog may be mentioned as the non-human animal.

(6) A kit for examining the classification of treatment and/or prevention of Th3 march-related diseases comprising an antibody against a cytokine and/or chemokine.
(7) A kit for deciding intension to use of treatment and/or prevention drug of Th3 march-related diseases comprising an antibody against a cytokine and/or chemokine.
(8) A kit for diagnosing Th3 march-related diseases comprising an antibody against a cytokine and/or chemokine.
(9) A kit for treatment and/or prevention of Th3 march-related diseases comprising an antibody against a cytokine and/or chemokine and the pharmaceutical composition according to one of claims 1 to 4.

In the kits (6)-(9), the antibody against a cytokine and/or chemokine may be carried on a bead array.

In the kits (6)-(9), Th3 march-related disease may be selected from at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3.

In the kits (6)-(9), the cytokine and chemokine may be at least one member selected from TGF-β1, IL-1β, IL-4, IL-5, IL-6, IL-9, IL-10, IL-13, IFN-γ, RANTES.

### BRIEF DESCRIPTION OF THE DRAWINGS

FIG. 1 is a schematic view showing one example of Th3 disease march, which is Th3 march based on examination of 797 dogs (in the figure, age (0-13 old) is dog's age). Similar Th3 march can be seen in examination of human (238 examples)

FIG. 2A is a schematic view showing zinc Th cell cytokine signal transmission.

FIG. 2B is an exploded view of FIG. 2A, which relates to early intervention of diseases by cytokine panel test with disease march, and shows relations among the relations of respective signals and diseases etc.

FIG. 2C is an exploded of FIG. 2A, which relates to early intervention of diseases by cytokine panel test with disease march, and shows relations among the relations of respective signals and diseases etc.

FIG. 2D is an exploded of FIG. 2A, which relates to early intervention of diseases by cytokine panel test with disease march, and shows a relation with cytokine used in the blood cytokine panel test.

FIG. 3 is a drawing showing the results of examining characteristic bone resorption in dog rheumatoid arthritis (examples of bone resorption diseases shifted from Th1 to Th2).

FIG. 4 shows a bone resorption image of arthritis due to rheumatoid arthritis which is Th1 vital reactive disease (post-treatment) when bone resorption and bone augmentation are balanced.

FIG. 5 is a drawing totally showing the cytokine panel test in clinically improved treatment examples.

FIG. 6 is a drawing showing the course of treatment of the severest atopic dermatitis (forearm).

FIG. 7 is a drawing showing the symptom of Ehlers-Danlos syndrome (EDS).

FIG. 8 is a drawing showing the finding of plain x-ray examination of EDS.

FIG. 9 is a drawing showing the finding of plain x-ray examination of Paget's disease of bone.

FIG. 10 is a drawing showing the course of treatment of Paget's disease of bone.

FIG. 11 is a drawing showing the course of treatment of EDS.

FIG. 12 is a drawing showing the finding of plain x-ray examination of the deterioration course of march asthma and at the time of treatment.

FIG. 13 is a drawing showing the course of treatment of the atopic dermatitis.

FIG. 14 is a drawing showing the finding of plain x-ray examination assuming Th march.

FIG. 15 is a drawing showing the construction of Znf (zinc finger) having DNA repair capacity.

FIG. 16 is a drawing showing the positional relationship of DNA repair capacity of Znf for the cytokine panel test for selecting a drug for atopic dermatitis or Th3 march disease.

FIG. 7 is a drawing of the calibration curve for quantitatively detecting filaggrin by ELISA method, prepared by using biotin-coupled polyclonal antibody which specifically coupled with filaggrin.

FIG. 18 is a drawing showing administration effect of zinc finger/Znf to a patient having filaggrin abnormality.

FIG. 19 is a drawing showing administration effect of zinc finger/Znf to a patient having filaggrin abnormality.

FIG. 20 is a drawing showing administration effect of zinc finger/Znf against Th1/Th2 march disease (plaque psoriasis).

FIG. 21 is a drawing showing the results of measurement of skin extension rate of dog Th3 march Th3 EDS-FLG by zinc finger/Znf.

FIG. 22 is a drawing showing the results of confirmation of an effect of shortening skin extension rate (improvement of EDS) upon dog Th3 march Th3 EDS-FLG by zinc finger/Znf.

FIG. 23 is a dental panorama image of a patient before and after treatment with the administration of zing finger/Znf, where the (upper and lower) left drawings shows images before treatment, the (upper and lower) right drawings shows images after treatment, and lower drawing are extended figures of the upper drawings (cut figures).

### EMBODIMENTS FOR CARRYING OUT THE INVENTION

The present invention will now be described in detail. The scope of the present invention is not restricted to the following description, and the present invention can be suitably modified without departing from the present invention.

The description includes the whole of Japanese Patent Application No. 2010-214531 filed on Sep. 24, 2010 which is the base of priority claiming. Also, all of the publications cited herein including prior art documents, laid-open patents, patent publications, are incorporated herein by reference.

### 1 Outline of the Invention

In the treatment of immune diseases, the correction of the immune balance expressed by the balance of Th1/Th2/Th3 cells is particularly important. It has hitherto been reported that specific cytokine and chemokine are increased in specific diseases, but there is no method for judging characteristics of disease and determining the administration of therapeutic agent or such by cytokine and chemokine panels.

Conventionally, an immune balance is generally measured by measuring intracellular cytokine with a flow cytometer. However, in this method, living lymphocytes are required, significantly restricting the testing scope. We make it possible to measure a balance of Th1/Th2/Th3 cells by measuring cytokine and chemokine of IL-1β, IL-4, IL-5, IL-6, IL-9, IL-10, IL-13, IFN-γ, RANTES, and GF-β1 in blood. Although trace amounts of cytokine and chemokine existing in a blood have hitherto been measured by an ELISA method, a considerable amount of blood is required in terms of measurement sensitivity from the viewpoint of handling and for measurement of various items of cytokine and thus, it has been very difficult to measure various items of cytokine and chemokine. We have discovered that the immune balance of each patient can be understood by measuring cytokine and chemokine of IL-1β, IL-4, IL-5, IL-6, IL-9, IL-10, IL-13, IFN-γ, RANTES, and TGF-β1 in the blood utilizing bead array which has been recently developed, to thereby makes an adequate intervention of medical treatment.

Specifically, clinical treatment of disease can be solved by high clinical examination technique and therapeutic agent (pharmaceutical composition) described later on or such. For example, in case of the atopy patient, the symptom of the patient will be changed with advancing years by allergy linkage phenomenon expressing atopic dermatitis at an infant period, bronchial asthma at a child period, and allergic rhinitis and conjunctiva inflammation at an adult period, as described as allergy march. It has also been known that the disease becomes server for example to be asthma. In recent years, it has been known that increase in severity can be inhibited by adequate intervention of medical treatment at the earliest stage, represented by the word “early intervention”.

The high clinical examination technique or such described above is also applicable to a treatment of disease deduced to take part in immune such as cancer and auto immune disease in addition to allergy. In case of specific diseases, such as pollinosis, asthma, and cancer, a kit which restricts items for a specific disease (cytokine etc.) among the panel can also be used for examination of treatment classification.

The present invention makes it possible to clarify an immune balance by measuring key cytokine utilizing panel containing various items of cytokine and others, and we have developed therapeutic or preventive drugs and treatment and prevention methods for correcting the immune balance. As a result, a treatment method, a treatment protocol and the like to meet the individual immune balance can be determined.

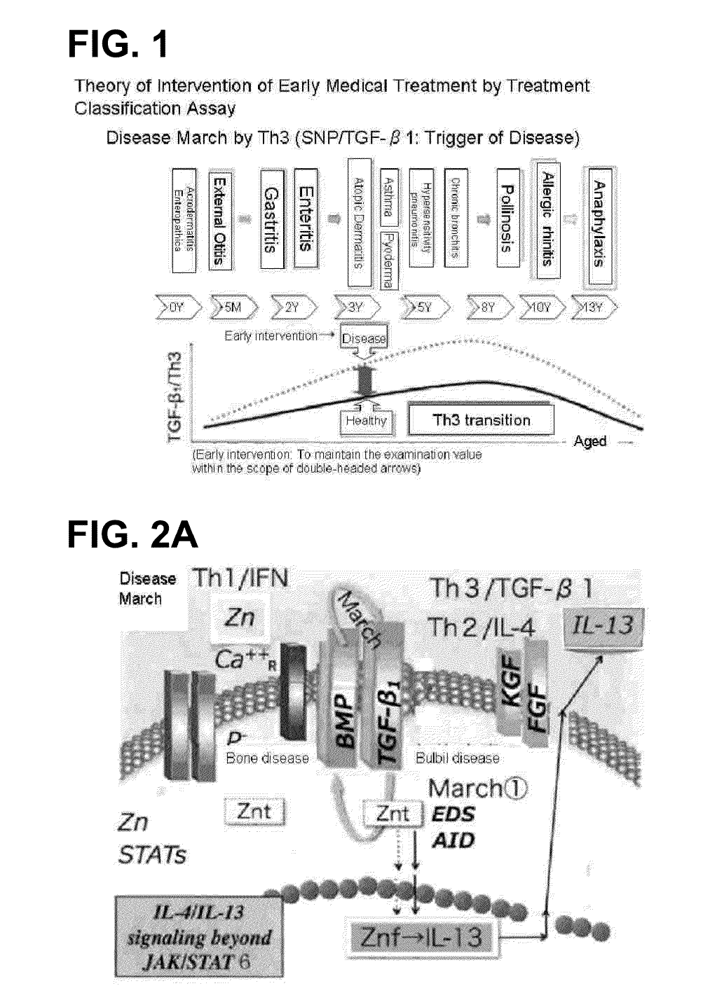

As a result, in the treatment of Th3 march-related diseases etc., an inspection method, treatment drug etc., for early intervention of the diseases have been accomplished. Although not restricted thereto, Th3 march includes a flow (linkage) of diseases as shown in FIG. 1. Such a clinical judgment system based on the present invention can be considered to make a huge contribution to the treatment of diseases deduced to have a relation to abnormality of the immune balance.

### 2. Pharmaceutical Composition

The pharmaceutical composition for treatment and/or prevention of Th3 march-related diseases comprises zinc as an essential ingredient, and preferably comprises zinc, calcium, and phosphor as essential ingredients. Also the pharmaceutical composition of the present invention preferably comprises pumpkin seeds (pumpkin seeds, matured pumpkin seeds) and corn silk, which are preferably heated.

In the pharmaceutical composition of the present invention, zinc (zinc agent) as an effective ingredient is preferably provided as metal zinc and various zinc compounds (such as zinc methionate), and yeast containing them (zinc yeast).

In the pharmaceutical composition of the present invention, the formulation ratio (weight ratio) of zinc, calcium and phosphor as effective ingredients is not specifically restricted, and for example, is preferably 2.5-3.5:1.5-2.5:0.5:1.5 (=zinc: calcium: phosphor), and particularly the formulation ratio is 3:2:1. The pumpkin seeds and corn silk are preferably formulated in a ratio similar to that of calcium.

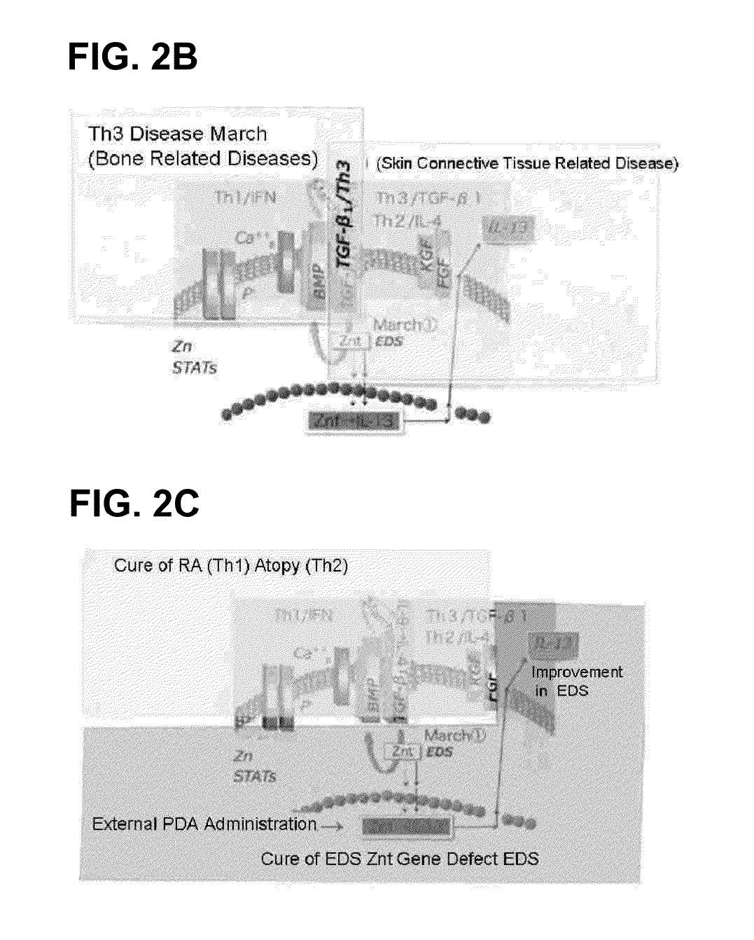

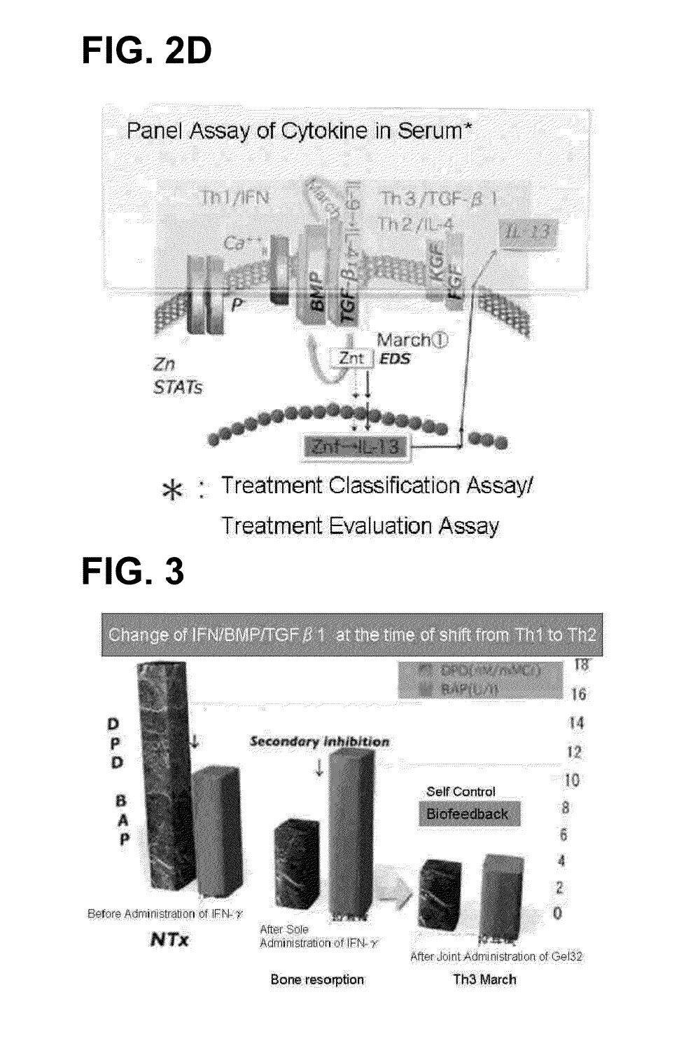

The pharmaceutical composition of the present invention activates intercellular zinc finger by incorporating zing as an effective ingredient into a cell by the action of calcium and phosphor. Specifically, incorporation of zinc in the cell activates DNA repair capacity by zinc finger (FIG. 2A and FIG. 2C). As shown in FIG. 2A and FIG. 2C, this activation can be confirmed by the enhancement of IL-13. The pharmaceutical composition of the present invention can treat, for example, Ehlers-Danlos syndrome, which is developed due to defect in gene of zinc transporter (Znt), by repairing the defect in gene as a result, as described later on.

The targets for treatment and prevention of Th3 march-related diseases by the pharmaceutical composition of the present invention include, but are not restricted to, at least one disease selected from among atopic dermatitis, Ehlers-Danlos syndrome, Paget's disease of bonem enteropathic acrodermatitis syndrome, external otitis, gastritis, enteritis, asthma, pyoderma, hypersensitivity pneumonitis, chronic tracheitis, pollinosis, allergic rhinitis and anaphylaxis, and also include cancer (such as bone cancer), autoimmune diseases (such as rheumatoid arthritis) and other diseases deduced to be taking part in immune.

The animals subjected to the administration of the pharmaceutical composition of the present invention (test animals) may be mammals which will develop various Th3 march-related diseases, and are not specifically restricted, including human, various non-human mammals. Examples of non-human mammals include dogs, cats, cows, horses, pigs, sheep, mice, rats, rabbits, marmots and hamsters, amongst them, dogs, cats, and horses are preferably, and dogs are particularly preferable.

The pharmaceutical composition of the present invention can be provided in a form including a pharmaceutically tolerable carrier in addition to the effective ingredients such as zinc. The term “pharmaceutically tolerable carrier” intended herein includes excipients, diluents, fillers, disintegrants, stabilizers, preservatives, buffers, emulsifiers, fragrances, colorants, sweetening agents, thickeners, stabilizing agents and other additives. By the use of at least one of such carries, various types of the pharmaceutical composition such as a capsule, an injection, a liquid, a suspension, an ointment, an emulsion or a syrup can be prepared. These pharmaceutical compositions can be administrated orally or parenterally. As administration forms, oral administration, intraoral administration, sublingual administration, gingival application, mucosal administration, aerosolized administration and the like can be mentioned. As parenteral administration forms, injections formulated according to the usual manner (injections for subcutaneous administration and injections for intravenous dose), transdermal administration (application), mucosal administration through nasal administration and aerosolized administration are included. The injection can be prepared by dissolving or suspending the pharmaceutical composition of the present invention in a pharmaceutically tolerable carrier such as physiological saline or commercially available distilled water. The pharmaceutical composition of the present invention is preferably administrated orally (specifically as described above), but not being restricted thereto.

Total content (proportion) of zinc, calcium, and phosphor (pumpkin seeds and corn silk are included if used) in the pharmaceutical composition of the present invention is not specifically restricted as long as it exhibits the effective for treating Th3 march-related diseases and is preferably from 10 to 100% by weight, more preferably 20 to 100% by weight, and most preferably 50 to 100% by weight.

The dosage of the pharmaceutical composition of the present invention is not specifically restricted, may differ from the diseases conditions, age, sex, weight of the patient, clinical conditions, therapeutic effects, administration methods, treating period, and may be suitably set by monitoring with the kit for testing classification of treatment or evaluating the transition of the syndrome or such.

Typically, as the dosage of the pharmaceutical composition of the present invention in the living body, the pharmaceutical composition of the present invention may be administrated in such an amount that zinc (Zn) concentration in the serum is not less than 55 μg/mL, preferably not less than 60 μg/mL, more preferably not more than 95 μg/mL, and most preferably the pharmaceutical composition of the present invention is administrated in the living body by suitably planning (unit dosage, average number of daily administration etc.) so that the endogenous vital reaction arrives at a normal value of not less than 0.88% and, at the same time, zinc concentration in the serum is not more than 60 μg/mL, and the dosage is not restricted. Particularly, in the case of dogs, the dosage is preferably in such an amount that zinc (Zn) concentration in the serum is not less than 85 μg/mL. The dosage is preferably carried out in the living body so that the zinc concentration in the serum does not exceed an upper limit of the normal value to avoid poisoning symptom.

Particularly, in the case where the mammals to be administrated is human, the dosage of the pharmaceutical composition of the present invention is preferably from 1 mg/kg weight to 10 g/kg weight, more preferably from 2 mg/kg weight to 2 g/kg weight, and most preferably from 2 mg/kg weight to 10 mg/kg weight as zinc per one administration. In the case where the mammals to be administrated is a dog or a cat, the dosage of the pharmaceutical composition of the present invention is preferably from 1 mg/kg weight to 10 g/kg weight, more preferably from 2 mg/kg weight to 2 g/kg weight, and most preferably from 2 mg/kg weight to 10 mg/kg weight as zinc per one administration.

Also, the present invention provides the use of zinc (Zn), calcium and phosphor (pumpkin seeds and corn silk are included, if they are used) (hereinafter referred to as “zinc etc.”) for production of pharmaceutical (drug) for treatment of Th3 march-related diseases. Also, the present invention provides zinc etc., for treatment of Th3 march-related diseases. Furthermore, the present invention provides a method for treating Th3 march-related diseases characterized by using zinc etc. Also, the present invention provides the use of zinc etc. for the treatment of Th3 march-related diseases.

### 3. Method for Treatment and Prevention

The pharmaceutical composition of the present invention can be used for treatment and prevention of Th3 march-related diseases. Specifically, a method for treating and/or preventing Th3 march-related diseases is provided which comprises: measuring a concentration of cytokine and/or chemokine in blood of a test animal; and initiating, continuing, interrupting or discontinuing an administration of the pharmaceutical composition of the present invention using the measured results as an index. Although not specifically restricted, as the cytokine and chemokine measured in the blood, at least one member selected from TGF-β1, IL-1β, IL-4, IL-5, IL-6, IL-9, IL-10, IL-13, IFN-γ, and RANTES can be preferably mentioned. In the present invention, it is also preferable to further measure the concentration of zing in blood.

The definitions of cytokines and chmokines including the convention contents and our discovered contents (shown by *) will be described.

TGF-β1: TGF-β1 is an anti-inflammatory cytokine which is produced by immune cells and cancer cells and which acts immunosuppressively. TGF-β1 suppresses proliferation and differentiation of lymphocytes (T cell and B cell) and suppresses the activation of NK cell. As a result, the immune response, inflammatory response, and hematopoiesis are suppressed. TGF-β1 is differentiated into Th17 in the presence of IL-6 or IL-4.
IL-1β: IL-1β is a cytokine produced from a monocyte, a macrophage, a B-lymphocyte, an endothelial cell, a keratinocyte and the like, and is also an endogenous pyrogen which acts on heat center of brain hypothalamus to causes a fever. IL-1β is a cytokine which activates T-lymphocyte to enhance the production of IL-2.
IL-4: IL-4 is a cytokine produced from activated CD4T cell (Th2 cell), CD8T cell, mast cell, basophil, and NKT cell. IL-4 is a cytokine which promotes the proliferation and the differentiation of Th2 cell, and a typical cytokine which regulates so-called humoral immunity. IL-4 acts on activated B-cell to promote class switching from IgM to IgG1, and IgE, and also promotes IgG1 antibody and IgE antibody. IL-4 antagonizes the action of IFN-γ to suppress the class switching to IgG2.
* Although atopy is a Th2 dominance disease, at and early stage of treatment, there is no relevance to improvement of clinical manifestation. The quantitative change in the allergy/atopy is not so large.
IL-5: IL-5 is a cytokine produced from Th2 cell or mast cell and promotes the proliferation of B-cells and production of antibody. IL-5 acts on eosinophilic progenitor cells to cause selective proliferation and differentiation of eosinophils.
* Whereas pruritus as complaint has sometimes heard from a patient of severe atopy from childhood at time of cure or immediately before healing, the function of IL-5 is the cause thereof.
IL-6: IL-6 is produced form monocyte-macrophage, vascular endothelial cells, fibroblasts, keratinocytes and the like. IL-6 proliferates B-cells and antibody-producing cells, and takes part in differentiation and activation of T cell, which produces IgG, IgM, and IgA (potentiation of antibody production). IL-6 acts upon hepatocyte to induce acute-phase proteins such as CRP and haptoglobin.
IL-9: In recent year, it has be clarified that IL-9 is secreted from T helper cells. This cell does not produce IL-4, IL-5, and IL-13, and solely produces IL-9 and IL-10. It has been reported that IL-9 promotes the proliferation of T-cell and mast cell and Th9 cell can be directly differentiated from Th2 cell induced from TGF-β or immature CD4+ cell from TGF-β and IL-4.
*) The production of IL-9 from Th9 cell in an atopy patient in a human or a dog is an essence of allergy and atopy. The quantitative production amount is high and is assumed to be a bio-parameter of severest atopy patient in a human or a dog (patient having an abnormality of skin over 36%) during the course of healing.
IL-10: IL-10 is mainly produced from Th2 cell and also can be produced from various cells such as monocyte, activated B-cell, and keratinocyte. IL-10 suppresses the production of IFN-γ from Th1 cell, and suppresses IL-1, IL-6, IL12, and TNF-α from macrophage.
IL-13: IL-13 is a cytokine which is mainly produced from Th2 cell and which can also be produced from NK cell and dendritic cell. IL-13 suppresses the differentiation and proliferation of B-cell, suppresses the production of proinflammatory cytokine of macrophage and acts on the expression of MH class II molecules. IL-13 acts on B-cell to promote T-cell dependent proliferation and class switching to IgE. Also, IL-13 suppresses the production of proinflammatory cytokine from monocyte, and enhances the production of IFN-γ.
* IL-13 activates zinc transporter (Znt) and zinc finger (Znf), and is a cytokine produced by Znf when PAD (zinc yeast-containing agent) is used. Also IL-13 is a cytokine produced when Znt deficiency is improved by the action of Znf in a Znt deficiency patient.
IFN-γ: IFN-γ is produced from Th1 cell of CD4T cell, CD8T cell, NK cell, and NKT cell: IFN-γ is mainly produced by CD4-positive helper T cell (particularly Th1 cell) and CD8 positive killer T-cell (CTL); and an NK cell and an NTK cell activated by IL-12 also produce IFN-γ.
Antiviral effect: IFN-γ has a function of enhancing cytotoxic activity of NK cell, CTL, and macrophage. IFN-γ increases the production of nitrogen monoxide (NO) by macrophage and promotes disinfection of intracellular eubacterium. IFN-γ promotes the expression of MHC class molecules. IFN-γ produced by Th1 cell suppresses the expression of CD40 ligand and suppresses the production of IgE antibody. IFN-γ produced by Th1 cell promotes differentiation of naive helper T-cell (Th0 cell) into Th1 cell, and suppresses the production of Th2 cell.
RANTES (Regulated upon Activation, Normal T cell Expressed and Secreted): RANTES is liberated from T-lymphocyte, eosinophil, macrophage, fibroblast, airway epithelial cell, mesangial cell, renal tubule epithelial cell, and the like. RANTES serves as eosinophil migration activity, enhancement of adhesive capacity and enhancement of production of active oxygen by eosinophil. Particularly, RANTES deeply involves accumulation and functions of T-cell in the field of allergic inflammation.

In the method for treatment and prevention of the present invention, the same description of section 2 can be applied to Th3 march-related diseases to be treated and prevented and test animals to be administrated.

In the method for treatment and/or prevention of the present invention, the concentrations of various cytokines and chemokines in blood are measured (hereinafter sometimes referred as “cytokine panel test”), based on the results of the measurement (specifically which kind of cytokine and the like and how much degree are expressed, enhanced, or suppressed), the presence or absence of the diseases at the present state and conditions thereof (pathology) of the test animal concerned are judged and also kinds of diseases considered to be developed in the next time as Th3 march are judged. Subsequently, based on the results of the judgment, the manner of changing the balance of Th1/Th2/Th3 is examined, and then the initiation, continuance, interruption or discontinuance of the administration of the pharmaceutical composition of the present invention described above is selected. Keeping, increasing or decreasing an administration period and an amount of administration are included in the initiation and continuance of the administration. Specifically, the pharmaceutical composition of the present invention is a composition for the treatment and/or prevention of Th3 march-related diseases, in addition to usage patterns of initiation and continuance of the administration as in the usual drug, the pharmaceutical composition of the present invention can select interruption and discontinuance of the administration to meet the results of cytokine panel test. In the case where in addition to curing the present disease, the prevention of the diseases excepted to be developed in the future is carried out at the same time (early intervention of diseases), while the administration should be contained in usual, there is a case that the administration is elaborately discontinued or interrupted in some cases. In this case, as a result, early intervention (intervention of early medical treatment) by DNA repair capacity (evaluated by IL-13) can be expected. This makes it possible decrease a period required for the treatment or such and the amount of drugs to be used.

Indication of values judge to be “high” in comparison with those of healthy subject with regard to concentrations of each cytokine in blood in cytokine panel test will be exemplified:

TGF-β1: not less than 5 ng/ml

IL-1β: not less than 1 pg/ml

IL-4: not less than 2 pg/ml

IL-5: not less than 2 pg/ml

IL-6: not less than 10 pg/ml

IL-9: not less than 30 pg/ml

IL-10: not less than 1 pg/ml

IL-13: not less than 2 pg/ml

IFN-γ: not less than 100 pg/ml

RANTES: not less than 2000 pg/ml.

In the present invention, a method for examining the classification of treatment and/or prevention of Th3 march-related diseases and a method for deciding intension to use of treatment and/or prevention drug are also provided, which comprises measuring a concentration of cytokine and/or chemokine in blood of a test animal (preferably also measuring the zinc concentration in blood) as described above; and deciding intension to use of treatment and/or prevention drug using the measured results as an index. The term “the classification of treatment and/or prevention” used herein is an examination to judge and anticipate the type of Th3 march-related diseases from the results of the cytokine panel test such as “what type of Th3 march-related diseases is developed in the present situation”, and “what type of Th3 march-related diseases will be developed in the future” and the like. The term “deciding intension to use of treatment and/or prevention drug” used herein is to decide necessity for using (administrating) the pharmaceutical composition of the present invention in order to keep the balance of Th1/Th2/Th3 cells in an adequate manner from the results of cytokine panel test, and the amount of administration, if used.

In the present invention, a method for diagnosing (detecting) Th3 march-related diseases is also provided, which comprises measuring a concentration of cytokine and/or chemokine in blood of a test animal (preferably also measuring the zinc concentration in blood) as described above; and diagnosing Th3 march-related diseases using the measured results as an index. Specifically from the results of the cytokine panel test, “what type of Th3 march-related diseases is developed in the present situation”, “what type of Th3 march-related diseases will be developed in the future”, and the state of the disease and pathology for the patient having the disease developed at the present state can be judged.

### 4 Kits

A kit for examining the classification of treatment and/or prevention of Th3 march-related diseases a kit for deciding intension to use of treatment and/or prevention drug of Th3 march-related diseases, a kit for diagnosing Th3 march-related diseases, and a kit for treatment and/or prevention of Th3 march-related diseases are provided in the present invention, which comprises an antibody against a cytokine and/or chemokine. These kits can make use of the cytokine panel test in various methods described previously in paragraph 3 “method”.

In the present invention, a kit for treatment and/or prevention of Th3 march-related disease comprising an antibody against cytokine and/or chemokine and the pharmaceutical composition of the present invention described above. As an embodiment of the application of the kit for treatment and/or prevention, the administration form is preferably decided and administrated by using the results of the measurement of the concentrations of various cytokines etc by the cytokine panel test utilizing the antibody as an index. When the kit is used as just mentioned, the kit can be used as a drug for treating and/or preventing Th3 march-related diseases. (The details are suitably applicable to the description of the method for treatment and/or prevention of Th3 march-related diseases).

Similarly, as for the applicable Th3 march-related diseases, cytokines, and chemokines, the description in paragraphs 2 and 3 are applicable.

In the kit of the present invention, the antibodies against various cytokine and/or chemokines are preferably carried on a bead array or such. When the bead array is used, the kit can be configured compactibly so that the concentrations of various types of cytokines from the collected blood can be simply detected all at once. In addition to the use of bead array, ELSA, Western blotting, immunochromatography (gold colloid method) can be used or jointly used with the bead array.

The kit of the present invention may include other constituents in addition to the antibodies against various cytokines and/or chemokines. Examples of other constituents include reagents for detecting a primary antibody, chromogenic substrates, various buffers, various tools and containers for collecting blood plasma, various containers which can be used in antigen-antibody reaction, and application manual. Specifically, in the case where the kit of the present invention is a kit utilizing the bead array, ELISA, or Western blotting, other constituents may include reagents for detecting a primary antibody, and chromogenic substrates. In the case of the kit utilizing immunochromatography (gold colloid method), in addition to gold colloid labeled antibodies and various solid phase antibodies, test sticks having a nitrocellulose membrane, a sample pad, or a conjugate can be mentioned.

The kit of the present invention may possess at least the antibody against various cytokines and/or chemokines described as a constituent. Consequently, the kit may or may not have all of the constituents used for diagnosing Th3 march-related diseases or such together with the antibody.

5. A method for evaluating DNA repair capacity of Znf (zinc finger) by being substituted with cytokines; and a method for selecting drugs by this evaluation method:

The construction of Znf (zinc finger) having DNA repair capacity is shown in FIG. 15.

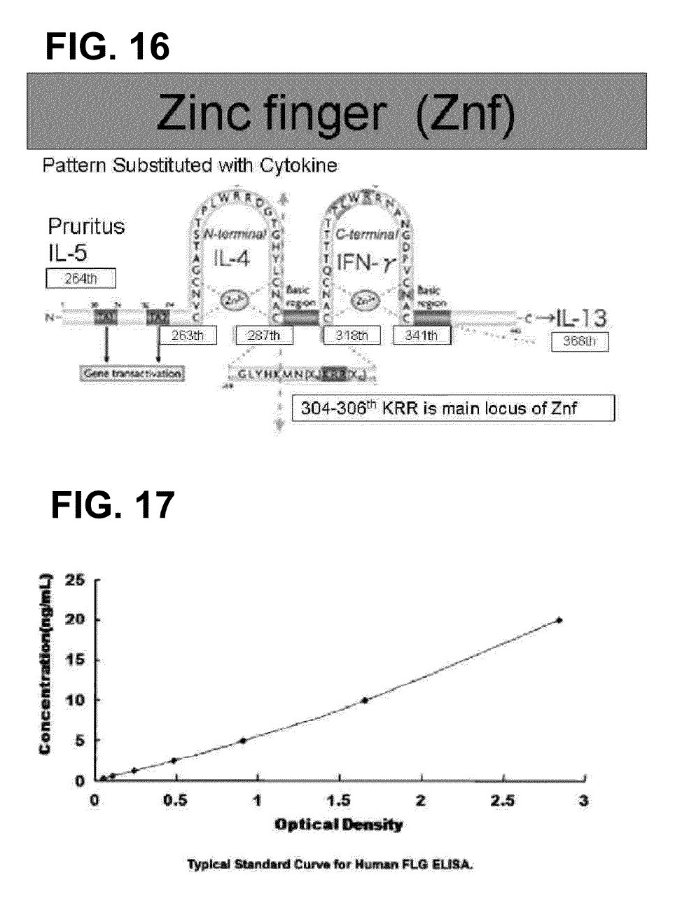

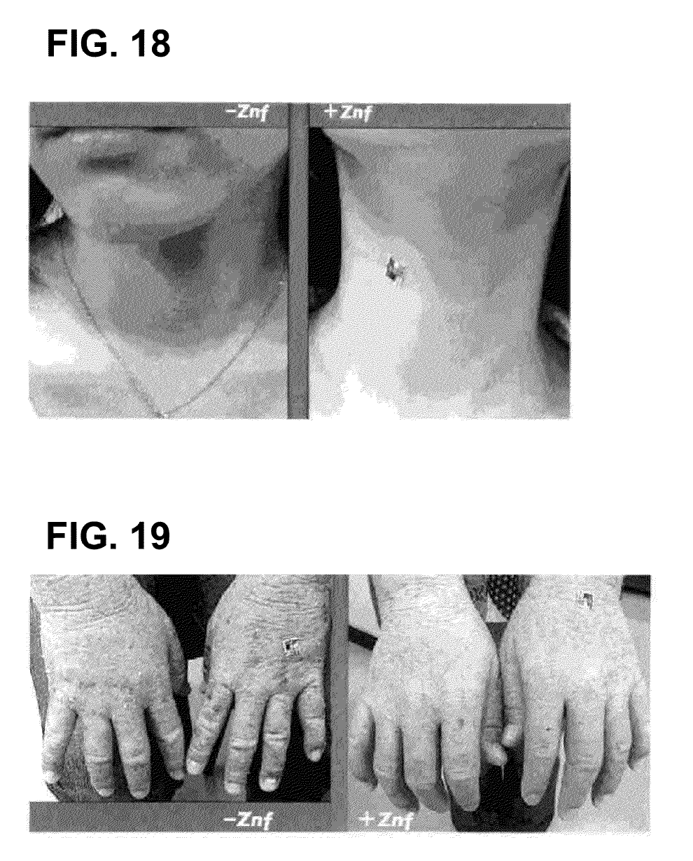

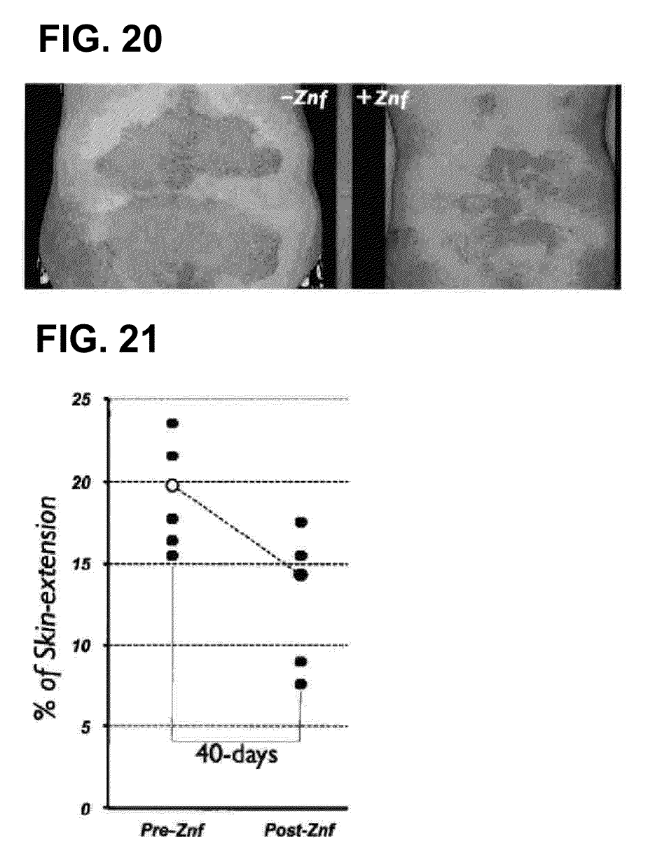

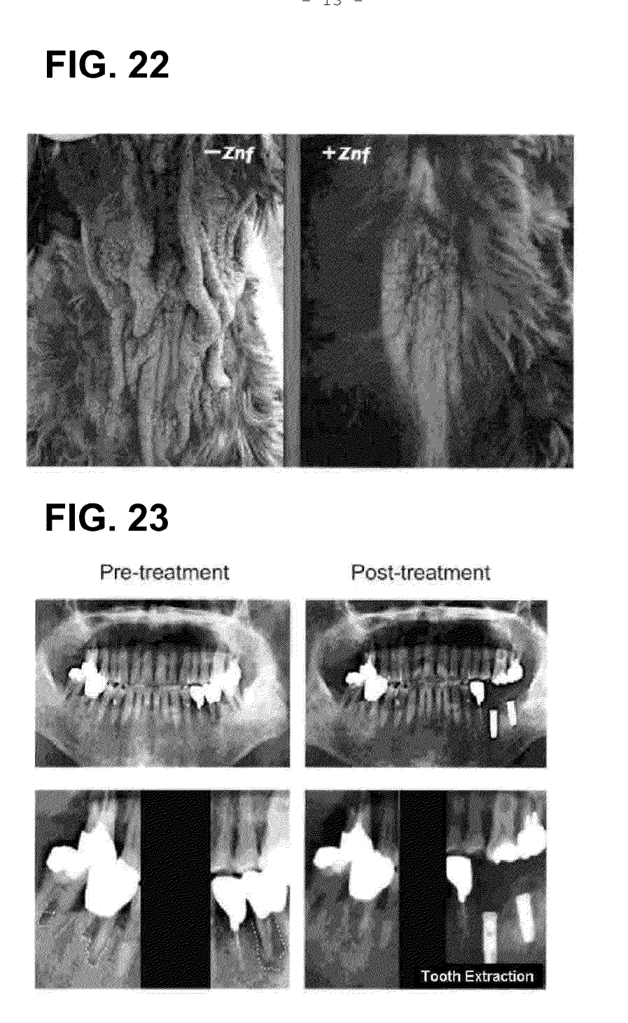

In order to measure the construction of FIG. 15 with the cytokine panel test including IL-13, the relation with the production of cytokine and amino acid sequence is clarified (FIG. 16: the pattern substituted with cytokine).

FIG. 16 is a drawing showing the positional relationship of DNA repair capacity of Znf for the cytokine panel test for selecting a drug for atopic dermatitis or Th3 march disease. IL-4 which is a Th2 cytokine relating to atopy exists on from 263^{rd} to 287^{th} loci. In interferon gamma (IFN-γ: mammals such as humans, dogs, and cats) which is a Th1 cytokine, the production region of the main locus exists on 318^{th} to 341^{st}, which is the C terminal.

In the IL-4 production region at the N terminal, the existence of IL-5 (mammals such as humans, dogs, and cats) or IL-13 on 264^{th} has been pointed out. The main locus of the DNA repair capacity is positioned from 304^{th} to 368^{th}, and as for the main locus of IL-13, which is a signal for activating Znf, the cytokine production region is positioned at 368^{th} in FIG. 16. The evaluation of various drugs represented by drug action at the time of the administration of zinc finger Znf/PAD can be performed from this relation (see Examples). The evaluation of the drug can also be performed by utilizing the cytokine panel test.

In turn, the translation period from N terminal to C terminal is about 6 weeks, making it possible to deduce the expression period of the drug action in an indirect manner. Also, this makes it possible to set the administration period of zinc finger Znf/PAD to atopic dermatitis patient (see Examples: Production of IL-13).

The present invention will be described in more detail by referring to the working examples, the present invention is not restricted the working examples.

### Example 1

### Early Intervention of Diseases Utilizing Th3 March

TABLE 1A
TGF-β1/TH3 March

TGF-β1/TH3 March
Skin Connective Tissue March
Zn Th Cell
Znt/Znf → Th9 → Th2 → Tfh → Th3 further taking part in Th17 and
Th22

TABLE 1B
Combs Classification Type IV in the context of Th3 March
March Coombs Classification Type IV

Type IVa: Reaction between Th1 cell and macrophage, and tuberculin
reaction, and contact dermatitis under Th1 vital reaction suppressed
condition are included herein
Type IVb Reaction between Th2 cell and eosinophil, bronchial asthma,
allergic rhinitis, protein-induced enteritis are included
Type IVc Reaction between CD8 and T cell and contact dermatitis is
included
Type IVd Reaction between T cell and neutrophil, and Behcet's disease
and the like are included.

TABLE 1C
Outline of Zinc Cell Cytokine Signal Transmission System

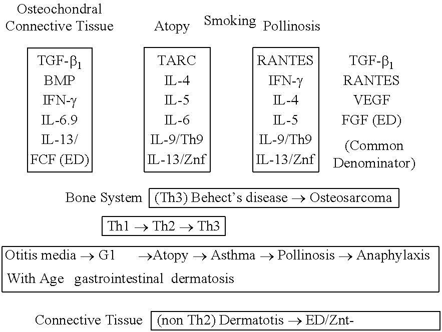

### DEFINITION AND OUTLINE

Th3 has made it clear for the first time by us. Th3 march via the signal transmission of zinc cell cytokine is controlled by calcium and phosphor transmitted to the cell and IFN and IL-13 (Znf) by means of cell surface BMP (bone morphogenetic protein) and TGF-β1 (newly defined as TGF-β1/Th3 march)

In clinically, the order seen in pathological change by age was relatively clear. Th3 march marches, from juvenile, Th1 vital reaction dependency (bone disease), Th2 vital reaction dependent atopy (skin disease as abnormality of connective tissue), and Th3 vital reaction (inhibitory factor of serous cure). The march thereafter marches bone disease or connective tissue disorder with advancing years. In this case, the disease is clinically developed, transiting from dog or human juvenile rheumatoid arthritis (Th1 bone tissue disease), atopic dermatitis (Th2 connective tissue disease) at 3 years old in both cases of dog and human, to Th3 disease. Th3 vital reactive disease shows the mixture of Th1 vital reaction with Th2 vital reaction. Since the vital reactive disease seen with age is mediated by zinc- or Znt-abnormality or deficiency (Ehlers-Danlos syndrome (EDS)), Th1 and Th2 reactive diseases become severe and psychiatric disorder (bipolar disorder type I and II, and delayed psychiatric disorder etc.) is complicated and for examples, severest atopy will be induced (see Examples). Clinically, it has been pointed out that bone cancer will be induced from bone Behcet's disease. An example of bone resorption diseases shifted from Th1 to Th2 is shown in FIG. 3.

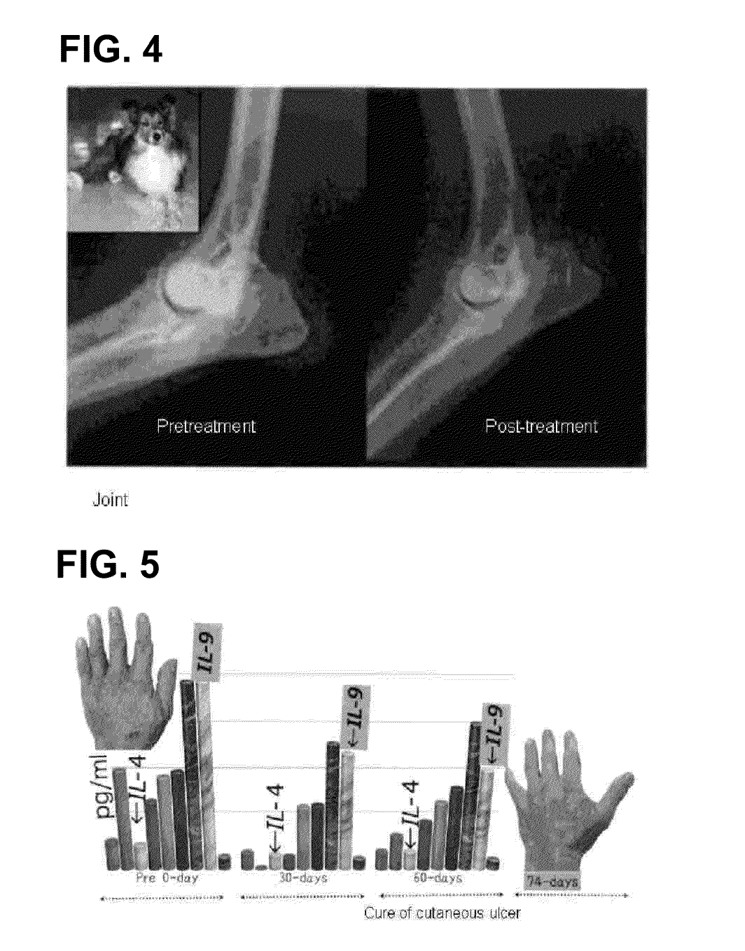

FIG. 3 is a drawing showing the results of examining characteristic bone resorption in dog rheumatoid arthritis. Th3 march via zinc cell cytokine signal is marching through a route of cell surface BMP and TGF-β1. During the course of the treatment of the dog with recombinant interferon-gamma (IFN-γ), bone resorption is predominant and on the other hand, examining bone resorption, it has been clinically discovered that BAP is mutually exclusively enhanced. When the healing can be seen thereafter (FIG. 4), bone resorption and bone augmentation are equilibrated. However, TH3 vital reaction is enhanced to clinically induce atopic dermatitiss.

In FIG. 4, according to the finding of the plain x-ray examination, a bone resorption image of arthritis due to rheumatoid arthritis which is Th1 vital reactive disease (pretreatment) is shown. In the right figure of FIG. 4 showing the post-treatment, an improvement image of joint was seen when bone resorption and bone augmentation were balanced. When the Th1 arthritis was cured (direct administration of 4MU of IFN-γ within the joint), hyperpermeability image due to invasion around the joint and inflammation of joint was significantly improved. According to the finding of post-treatment in the right figure of FIG. 4, incisura trochlearis in the elbow joint was visualized and significant improvement tendency was observed (Evaluation: While the dog before treatment hated going for a walk, the dog could go for a walk (about 5 minutes). It is noted that the standard walking period in the same kind of dog is within 30 minutes).

### Example 2

### Institution of Cytokine Panel Test Against Atopic Dermatitis

### 1. Cytokine Panel of Healthy Subject

A cytokine panel test was carried out for 248 subjects approved to be healthy subjects, and a cytokine panel of 63 volunteers in which persons clinically suggested (smoking (20 cigarettes/day) and pollinosis) and the elderly were excluded, are shown in the following Table 1D. From this Table, characteristics of each disease (severest atopic dermatitis) were separately prepared as a patient's cytokine panel (similar cytokine panel test was prepared for animals such as dogs).

TABLE 1D

| | | | TGF | | | | | | | | | | IL-9/ | | |
|---|---|---|---|---|---|---|---|---|---|---|---|---|---|---|---|
| No. | Sex | Age | ng/ml | PDGF | Il-1b | IL-1ra | IL-2 | IL-4 | IL-5 | IL-6 | IL-7 | IL-8 | Th9 | IL-10 | IL-12 |
| 1 | M | 64 | 0.0 | 35.14 | 0.45 | 57.34 | 1.03 | 0.59 | 1.25 | 2.59 | 1.62 | 6.33 | 3.26 | 0.34 | 0.92 |
| 2 | M | 62 | 0.0 | 3.17 | 0.52 | [symbol] | 1.06 | 0.52 | 1 | 4.24 | 1.62 | 7.65 | 2.74 | 0.94 | 2.81 |
| 22 | F | 36 | 1.0 | [symbol] | [symbol] | 170.3 | [symbol] | [symbol] | 1.78 | 5.36 | 4.44 | 10.51 | 11.6 | 1.88 | 12.71 |
| 23 | F | 55 | 3.3 | 161.38 | 1.31 | 151.53 | 2.91 | 1.04 | 1.4 | [symbol] | [symbol] | 6.07 | 14.14 | 1.16 | 3.52 |
| 24 | F | 35 | 0.7 | 230.26 | [symbol] | 87.43 | 4.89 | 1.42 | 1.78 | 5.96 | 2 | 21.72 | 12 | 0.28 | 3.43 |
| 28 | F | 35 | 1.2 | 83.64 | 0.79 | 74.52 | 0.25 | 0.77 | [symbol] | 2.59 | 2.22 | 6.43 | 12.92 | 0.61 | 3.92 |
| 33 | F | 40 | 4.6 | 673.62 | [symbol] | 191.46 | 2.29 | 1.26 | 1.56 | 5.4 | 2.79 | 7.08 | 8 | 1.13 | 4.76 |
| 34 | F | 44 | 0.00 | 42.86 | 1.24 | 101.57 | 1.26 | 1.07 | 0.98 | 2.59 | 3.31 | 7.17 | [symbol] | 0.7 | 4.76 |
| 35 | F | 31 | 2.2 | 162.84 | 1.15 | 116.84 | 0.45 | 1.02 | 0.7 | 4.02 | 3.04 | 8.77 | 10.5 | 0.46 | 5.41 |
| 36 | F | 31 | 1.0 | 103.77 | 1.66 | 172.85 | [symbol] | 1.47 | 0.78 | [symbol] | 35.3 | 10.49 | 4.24 | 0.99 | 6.12 |
| 37 | F | 40 | 2.0 | 369.39 | 1.34 | 160.95 | 10.51 | 1.45 | 1.3 | [symbol] | 3.63 | 8.87 | 8.27 | 1.29 | 7.64 |
| 39 | F | 36 | 0.7 | [symbol] | 0.39 | 52.95 | 10.6 | 0.65 | 0.97 | 2.48 | 1.34 | 3.53 | 2.84 | 0.39 | 3.3 |
| 45 | F | 40 | 3.0 | 1638.91 | 1.78 | 180.36 | 9.1 | 1.65 | 1.56 | 6.54 | 3.74 | 9.52 | 13.14 | 0.4 | 8.58 |
| 48 | F | 26 | 1.4 | 84.47 | 0.69 | 81.8 | 14.6 | 0.62 | 1.76 | 2.3 | 1.78 | [symbol] | 1.42 | 0.36 | 1.96 |
| 50 | F | 33 | 2.2 | 356.37 | 1.69 | 133.21 | 0.56 | 1.7 | [symbol] | 5.05 | 3.83 | 10.51 | 5.85 | 1.03 | 7.53 |
| 51 | F | 54 | 2.9 | 216.61 | 1.32 | 113.55 | 0.45 | 1.14 | 1.87 | 4.27 | 3.06 | 7.7 | [symbol] | 0.04 | [symbol] |
| 57 | M | 32 | 1.2 | [symbol] | [symbol] | 102.14 | 0.38 | 1.02 | 1.78 | 4.3 | 3.31 | 6.05 | 4.73 | 0.21 | 4.87 |
| 60 | F | 21 | 0.5 | 268.86 | 0.74 | 76.2 | [symbol] | 0.35 | 0.9 | 2.9 | 4.3 | 7.33 | 3.42 | 0.5 | 3.52 |
| 61 | F | 27 | 0.2 | 213.94 | 1.27 | 148.4 | 7.68 | 1.3 | 1.56 | 4.98 | 2.74 | 10.52 | 12.04 | 0.98 | 7.66 |
| 62 | F | 35 | 2.1 | 475.97 | | | 16.53 | 1.84 | 1.76 | 7.31 | 5.47 | 12.65 | 21.51 | 1.45 | 14.34 |
| 63 | F | 46 | 3.7 | 518.9 | 1.9 | 169.13 | 5.25 | 1.19 | 1.64 | 6.17 | [symbol] | 10.86 | 11.7 | 0.53 | 17.06 |
| 64 | F | 24 | 2.0 | 2313.21 | | | 15.73 | | 0.8 | 5.8 | [symbol] | [symbol] | 13.8 | 1.42 | 15.4 |
| 65 | F | 26 | 1.0 | 615.16 | | | 16.38 | | 0.98 | [symbol] | 6.93 | [symbol] | 14.2 | | 10.48 |
| 66 | F | 67 | [symbol] | [symbol] | [symbol] | 129.83 | | 1.28 | 0.67 | 4.49 | 3.26 | 10.18 | 6.11 | 0.35 | 8.77 |
| 67 | M | 47 | 0.2 | 46.17 | 1.01 | 140.02 | | 0.92 | 1.76 | [symbol] | 3.51 | 6.13 | 20.3 | 0.23 | 3.27 |
| 73 | M | 23 | 3.9 | 1025.11 | 1.97 | 137.71 | 7.33 | 1.48 | 1.98 | [symbol] | 2.52 | 9.01 | 9.53 | 1.16 | 5.5 |
| 74 | M | 23 | 2.0 | 2181.0 | | | | | [symbol] | | 6.61 | 20.53 | 25.91 | 1.67 | 6.45 |
| 75 | M | 24 | 2.2 | 292.39 | 1.08 | [symbol] | 0.03 | 1.39 | [symbol] | 4.14 | [symbol] | [symbol] | [symbol] | 0.79 | 7.95 |
| 76 | F | 24 | 2.6 | 616.62 | 1.17 | 126.17 | 5.63 | [symbol] | 1.9 | 4.27 | 2.3 | 7.63 | 8.63 | [symbol] | 8.34 |
| 77 | F | 30 | 1.2 | 310.51 | | 130.2 | 7.65 | 1.93 | 1.76 | | 3.2 | 9.2 | 4.68 | 0.65 | 3.95 |
| 97 | F | 41 | 1.6 | 570.8 | 0.78 | 30.68 | 5.57 | 1.02 | 1.98 | 4.02 | 2.25 | 4.75 | 3.26 | 0.18 | 3.24 |
| 98 | F | 50 | 0.4 | 370.68 | 0.95 | [symbol] | 8.43 | 0.9 | [symbol] | 2.95 | [symbol] | 6.73 | 4.1 | 0.48 | 4.32 |
| 99 | F | 25 | 1.9 | 433.01 | 1.81 | 155.12 | 7.26 | [symbol] | 1.52 | [symbol] | 2.65 | 9.52 | 10.72 | 0.88 | 10.24 |
| 101 | F | 38 | 0.0 | 1156.2 | 0.67 | 144 | 3.98 | 1.86 | 1.89 | | 3.78 | 13.34 | 11.78 | 0.61 | 5.43 |
| 102 | F | 45 | 0.4 | 1761.05 | 0.69 | 155.7 | 14.84 | 1.92 | 1.54 | 7.04 | 4.17 | 15.73 | 12.79 | 0.76 | 6.54 |
| 104 | M | 21 | 0.2 | 2504.05 | 0.67 | 156.8 | 14.32 | 1.87 | 1.76 | 9.87 | 5.75 | 17.06 | 20.64 | 0.78 | 5.43 |
| 107 | F | 70 | 0.6 | 108.58 | [symbol] | 99.3 | 4.56 | 1.13 | 1.58 | 5.48 | 3.16 | 7.93 | 5.77 | 0.12 | 4.5 |
| 110 | F | 48 | 2.0 | 1781.4 | 0.67 | 58.7 | 16.54 | 0.37 | 1.76 | 8.89 | 5.3 | 14.36 | 5.67 | 0.78 | 17.66 |
| 111 | F | 39 | 0.0 | 89.07 | 0.92 | 71.8 | 4.83 | 0.89 | 1.65 | 4.86 | 1.93 | 7.24 | 11.41 | 0.45 | [symbol] |
| 112 | F | 39 | 0.7 | 239.5 | [symbol] | 174.41 | 3.51 | [symbol] | [symbol] | 9.05 | 4.39 | 11.74 | 11.13 | 1.49 | 7.32 |
| 114 | F | 45 | 2.7 | 736.35 | 1.94 | 176.64 | 2.19 | 1.88 | [symbol] | 5.57 | 3.69 | 11.7 | 7.32 | 1.74 | 9.8 |
| 118 | F | 33 | 3.8 | 453.37 | [symbol] | 165.43 | 8.23 | 1.73 | [symbol] | 6.08 | 3.55 | 9.15 | 8.35 | 1.16 | [symbol] |
| 119 | F | 33 | 2.5 | 487.43 | [symbol] | 143.54 | 14.78 | 0.55 | 0.54 | 9.5 | 6.05 | 13.84 | 11.96 | 1.76 | 11.57 |
| 122 | M | 47 | 0.2 | 1087.48 | 1.87 | [symbol] | 10.67 | 0.55 | 1.98 | 8.26 | 4.53 | 15.3 | 18.31 | 1.69 | 11.4 |
| 123 | F | 54 | 1.0 | 885.97 | 1.85 | 187.33 | 8.09 | 1.54 | [symbol] | 6.9 | 3.75 | 15.44 | 11.49 | [symbol] | 12.92 |
| 124 | F | 58 | 1.8 | 185.37 | 1.91 | 134.32 | [symbol] | 1.41 | 1.54 | 4.6 | 4.14 | 9.43 | 5.95 | 1.08 | 4.14 |
| 125 | F | 36 | 1.8 | 283.85 | 1.72 | 158.62 | 4.56 | 1.28 | 0.67 | 5.1 | 3.65 | 9.56 | 12.04 | 1.27 | 5.79 |
| 128 | F | 66 | 1.0 | 1120.8 | 1.89 | [symbol] | 13.95 | 0.54 | 0.96 | 9.38 | 6.02 | 16.72 | 19.26 | 1.56 | [symbol] |
| 129 | M | 31 | 0.3 | 694.47 | 1.47 | 142.92 | 8.02 | 1.48 | 0.87 | 4.76 | 14.3 | 10.02 | 6.03 | 1.03 | 7.69 |
| 135 | M | 72 | 0.0 | 34.59 | 1.57 | 165.04 | 5.18 | 1.11 | 0.76 | 4.14 | 31.15 | 8.24 | 7.65 | 0.89 | 5.33 |
| 136 | M | 70 | 0.4 | 3227.7 | 1.63 | 156.05 | 5.19 | 1.73 | 0.59 | 6.94 | [symbol] | 12.03 | 10.64 | 1.5 | 15.75 |
| 139 | M | 28 | 2.6 | 379.09 | 1.87 | 167.03 | 5.43 | 0.87 | 1.99 | 1.76 | 4.59 | 14.17 | 6.24 | 1.62 | 12.33 |
| 152 | F | 24 | 1.0 | 408.1 | [symbol] | 111.83 | 1.36 | [symbol] | 0.57 | 3.06 | 3.2 | 6.24 | 8.43 | 0.7 | 3.73 |
| 154 | F | 21 | 0.7 | 183.29 | 1.68 | 174.99 | 5.84 | 1.6 | 0.76 | 5.11 | 3.67 | 11.32 | 5.45 | 1.18 | 3.76 |
| 155 | F | 20 | 0.6 | 100.41 | 1.71 | [symbol] | 2.94 | 1.07 | 0.67 | 4.25 | 4.38 | 8.31 | 11.08 | [symbol] | 4.34 |
| 157 | F | 23 | 4.5 | 849.15 | 1.37 | 178.75 | 2.76 | 1.9 | 1.87 | 9.35 | 3.74 | 12.88 | 9.28 | 1.21 | 11.67 |
| 168 | F | 26 | 0.0 | 298.51 | 0.95 | 34.61 | 2.54 | 0.76 | 1.56 | 5.98 | 2.52 | 7.05 | 3.37 | [symbol] | 2.94 |
| 31 | F | 32 | 0.2 | 99.07 | 1.23 | [symbol] | 2.08 | 1.08 | 1.54 | 3.43 | 2.81 | 8.03 | 3.13 | 0.67 | 2.05 |
| 47 | M | 36 | 4.2 | 274.31 | 1.23 | 102.71 | 2.06 | [symbol] | 0.89 | 3.48 | 3.02 | 6.98 | 4.41 | 0.52 | [symbol] |
| 48 | M | 33 | 1.0 | 4561.6 | 1.45 | 112.4 | 3.87 | 1.47 | 0.76 | 4.99 | 8.13 | 10.47 | 9.11 | 1.39 | [symbol] |
| 132 | F | 48 | 1.0 | 396.22 | 1.45 | [symbol] | 2.78 | 1.47 | 0.98 | [symbol] | 4.84 | 13.57 | 4.32 | 1.87 | 5.43 |
| 134 | F | 78 | 0.3 | 507.19 | 1.89 | 116.67 | 5.98 | 1.73 | 1.67 | [symbol] | 4.21 | 11.27 | 8.48 | 1.27 | 9.56 |
| 156 | F | 35 | 0.2 | 1390.57 | 1.71 | [symbol] | 4.28 | 1.26 | [symbol] | 5.42 | 2.85 | 11.45 | 7.81 | 1.43 | 7.12 |
| | L13/ | | IL-17/ | | | | GM- | | | MCP- | MIP- | MIP- | | | |
| No. | Znf | IL-15 | Th17 | Eotaxin | FGF | G-CSF | SCF1 | INF-? | IP-10 | 11 | 1a | 1b | ANTES | TNF-a | VEGF |
| 1 | 1.35 | 0.5 | 0.56 | 36.78 | 6.68 | 8.04 | 11.9 | 13.06 | 166.54 | 35.72 | 0.61 | 47.4 | 1707.36 | 7.12 | 0 |
| 2 | 3.59 | 0.5 | 0.67 | 37.87 | 6.57 | 5.67 | 14.56 | 19.17 | 166.76 | 30.87 | 0.69 | 47.94 | 1506.92 | 4.04 | 3 |
| 22 | 6.53 | 4.25 | 0.76 | 43.28 | 41.04 | 6.54 | 33.21 | 54.87 | 144.32 | 10.7 | 3.1 | 38.56 | 1370.32 | 28.49 | 10 |
| 23 | 4.79 | 0.5 | 3.07 | 34.57 | 10.34 | 5.2 | 15.87 | 44.01 | 153.44 | 15.9 | 1.24 | 28.73 | 1700.74 | 12.92 | 0 |
| 24 | 2.55 | 0.5 | 7.55 | 44.73 | 28.64 | 6.54 | 34.83 | 75.34 | 191.52 | 17.03 | 1.5 | 29.81 | 1044.16 | 21.04 | 2 |
| 28 | 5.64 | 0.5 | 0.1 | 37.86 | 5.08 | 4.47 | 12.93 | 27.23 | 236.63 | 12.88 | 0.81 | 30.61 | 1479.94 | 7.71 | 2 |
| 33 | 6.36 | 0.5 | 5.24 | 36.84 | 6.54 | 5.87 | 20.17 | 48.63 | 195.48 | 9.65 | 1.56 | 35.81 | 1416.79 | 14.94 | |
| 34 | 4.16 | 0.5 | 5.55 | 19.29 | 24.37 | 4.32 | 17.68 | 33.45 | 166.54 | 3.2 | 2.22 | 35.53 | 1032.11 | 10.58 | 0 |
| 35 | 5.58 | 0.5 | 0.13 | 27.32 | 1.84 | 3.54 | 16.2 | 34.14 | 195.81 | 16.38 | 1.15 | 22.03 | 1625.14 | 12.41 | 2 |
| 36 | 3.75 | 0.5 | 7.02 | 30.65 | 10.77 | 4.32 | 23.67 | 51.17 | 208.04 | 10.21 | 1.29 | 22.54 | 1540.29 | 17.99 | 5 |
| 37 | 2.04 | 2.82 | 19.44 | 23.54 | 31.48 | 3.9 | 30.58 | 58.21 | 297.95 | 17.45 | 2.31 | 37.58 | 1377.98 | 19.51 | 1 |
| 39 | 2.78 | 0.6 | 1.07 | 30.55 | 8.76 | 4.64 | 8.43 | 15.44 | 118.76 | 3.48 | 1.6 | 34.2 | 1334.54 | 1.73 | 3 |
| 45 | 7.18 | 4.31 | 0.75 | 19.79 | 47.54 | 6.55 | 37.74 | 56.53 | 200.54 | 15.43 | 3.4 | 62.73 | 1630.25 | 25.47 | 2 |
| 48 | 2.31 | 0.5 | 0.1 | 15.22 | 4.32 | 4.4 | 11.96 | 18.43 | 268.34 | 11.45 | 1.06 | 24.57 | 595.99 | 2.55 | 2 |
| 50 | 3.71 | 0.5 | 2.41 | 12.11 | 16.68 | 5.43 | 21.41 | 62.3 | 196.02 | 0.3 | 1.01 | 19.38 | 1389.19 | 18.34 | 2 |
| 51 | 0.06 | 0.5 | 4.61 | 25.92 | 9.12 | 8.53 | 13.58 | 36.34 | 162.1 | 9.45 | 1.09 | 28.49 | 1419.63 | 10.39 | 4 |
| 57 | 7.26 | 0.5 | 0.1 | 43.21 | 41.54 | 8.76 | 14.07 | 33.43 | 209.04 | 19.01 | 0.12 | 25.34 | 1370.92 | 8.54 | 5 |
| 60 | 2.59 | 0.5 | 2.67 | 32.05 | 33.21 | 8.9 | 18.34 | 24.59 | 167.65 | 15.26 | 0.69 | 44.73 | 1405.42 | 7.37 | 6 |
| 61 | 8.05 | 0.5 | 13.11 | 23.76 | 26.84 | 6.54 | 27.11 | 49.98 | 221.77 | 29.48 | 3.35 | 33.61 | 1300.93 | 10.56 | |
| 62 | 12.46 | 7.03 | 13.2 | 34.51 | 27.59 | 8.76 | 41.17 | 65.44 | 228.54 | 19.11 | 2.91 | 31.27 | 1273.67 | 25.3 | 5 |
| 63 | 11.36 | 1.06 | 14.37 | 21.54 | 33.65 | 6.54 | 36.54 | 52.78 | 225.43 | 17.37 | 6.09 | 18.59 | 1064.26 | 24.78 | 6 |
| 64 | 5.94 | 7.35 | 16.7 | 23.56 | 44.32 | 5.43 | 32.43 | 92.56 | 262.52 | 16.06 | 4.94 | 31.84 | 659.77 | 24.54 | 9 |
| 65 | 6.26 | 2.41 | 13.71 | 40.22 | 48.52 | 7.65 | 43.75 | 83.41 | 229.52 | 11.81 | 9.18 | 37.68 | 1355.1 | 29.54 | 4 |
| 66 | 5.56 | 0.5 | 2.45 | 40.21 | 3.44 | 7.75 | 19.75 | 38.36 | 231.46 | 22.53 | 1.01 | 11.78 | 1176.87 | 9.72 | 6 |
| 67 | 1.4 | 0.5 | 0.1 | 33.32 | 7.65 | 7.65 | 18.18 | 39.2 | 191.38 | 14.25 | 1.65 | 23.40 | 881.35 | 3.21 | 5 |
| 73 | 5.58 | 3.37 | 8.45 | 34.35 | 13.38 | 9.36 | 33.3 | 57.26 | 225.77 | 28.16 | 1.57 | 39.33 | 1564.9 | 16.54 | 6 |
| 74 | 11.51 | 0.98 | 7.65 | 43.71 | 15.43 | 7.65 | 44.32 | 44.54 | 225.43 | 17.47 | 5.49 | 44.17 | 1121.95 | 15.43 | 18 |
| 75 | 8.93 | 0.5 | 4.77 | 41.67 | 14.82 | 3.02 | 23.5 | 34.14 | 271.78 | 22.5 | 1.52 | 47.23 | 1249.23 | 11.91 | 5 |
| 76 | 3.10 | 0.5 | 2.46 | 40.76 | 11.22 | 6.87 | 22.83 | 38.7 | 161.52 | 24.25 | 1.85 | 20.89 | 1584.08 | 11.82 | 8 |
| 77 | 3 | 0.98 | 2.87 | 25.54 | 11.76 | 6.64 | 36.54 | 43.2 | 251.02 | 21.95 | 1.49 | 50.71 | 1184.87 | 29.22 | 4 |
| 97 | 2.31 | 0.5 | 0.1 | 16.52 | 14.65 | 8.28 | 11.96 | 22.8 | 251.38 | 16.38 | 0.82 | 48.32 | 1650.11 | 7.04 | 0 |
| 98 | 3.19 | 0.5 | 0.1 | 31.27 | 4.27 | 7.16 | 13.26 | 31.23 | 170.94 | 13.04 | 0.3 | 17.39 | 1374.04 | 9.22 | 1 |
| 99 | 7.08 | 3.96 | 9.35 | 32.57 | 22.53 | 7.36 | 24.84 | 44.68 | 294.97 | 12.8 | 2.77 | 25.72 | 1490.22 | 14.27 | 5 |
| 101 | 8.76 | 5.43 | 7.65 | 40.21 | 15.43 | 7.86 | 16.64 | 85.37 | 197.43 | 34.04 | 6 | 33.88 | 1335.17 | 16.75 | 1 |
| 102 | 10.27 | 7.65 | 6.64 | 37.87 | 16.54 | 9.93 | 44.79 | 71.65 | 128.25 | 17.99 | 5.99 | 25.37 | 1449.44 | 21.72 | 28 |
| 104 | 9.11 | 7.96 | 3.67 | 25.76 | 16.54 | 7.54 | 48.51 | 82.65 | 176.67 | 28.41 | 4.34 | 30.7 | 1183.5 | 21.34 | 26 |
| 107 | 4.08 | 0.5 | 1.66 | 26.75 | 37.65 | 5.68 | 15.55 | 37.02 | 178.54 | 33.55 | 0.73 | 27.01 | 1493.92 | 11.32 | 1 |
| 110 | 10.05 | 7.06 | 4.32 | 26.54 | 49.61 | 9.12 | 16.78 | 78.4 | 165.43 | 22.59 | 3.42 | 70.36 | 667.35 | 23.45 | 14 |
| 111 | 16.36 | 0.5 | 3.79 | 25.52 | 2.83 | 6.21 | 17.35 | 27.23 | 165.43 | 15.28 | 1.06 | 43.34 | 1511.8 | 1.9 | 2 |
| 112 | 17.5 | 0.5 | 8.93 | 28.49 | 21.76 | 8.53 | 30.07 | 60.58 | 178.9 | 40.39 | 2.14 | 22.49 | 1311.24 | 13.59 | 13 |
| 114 | 6.35 | 0.79 | 12.79 | 7.03 | 25.58 | 7.19 | 26.02 | 51.98 | 175.6 | 15.4 | 1.96 | 25.11 | 1125.84 | 18.21 | 12 |
| 118 | 4.38 | 0.5 | 10.31 | 4.25 | 18.74 | 7.65 | 26.02 | 52.62 | 235.6 | 16.59 | 1.74 | 184.2 | 1347.18 | 23.45 | 14 |
| 119 | 7.31 | 0.54 | 18.34 | 41.92 | 33.47 | 6.54 | 43.24 | 67.53 | 270.85 | 16.35 | 3.59 | 22.76 | 1315.11 | 21.72 | 10 |
| 122 | 7.88 | 6.24 | 8.76 | 41.5 | 39.18 | 9.14 | 37.91 | 65.29 | 212.43 | 30.92 | 2.94 | 26.16 | 1214.24 | 14.6 | 9 |
| 123 | 6.26 | 3.07 | 7.65 | 43.21 | 40.48 | 7.14 | 25.18 | 58.63 | 235.93 | 39.24 | 2.62 | 46.13 | 1385.71 | 17.8 | 15 |
| 124 | 5.99 | 0.5 | 8.77 | 48.6 | 12.75 | 5.46 | 23.84 | 41.2 | 216.15 | 18.90 | 1.55 | 52.67 | 1542.15 | 23.32 | 4 |
| 125 | 6.54 | 0.5 | 9.73 | 48.12 | 17.25 | 9.09 | 20.67 | 36.61 | 266.52 | 28.04 | 1.38 | 32.52 | 1524.58 | 17.4 | 3 |
| 128 | 11.75 | 8.62 | 12.65 | 48.09 | 16.55 | 9.68 | 47.47 | 77.94 | 219.74 | 17.48 | 4.38 | 31.71 | 563.18 | 15.7 | 21 |
| 129 | 5.68 | 0.5 | 9.58 | 14.79 | 30.14 | 6.84 | 21 | 42.35 | 211.64 | 15.82 | 2.48 | 27.28 | 1571.69 | 17.48 | 2 |
| 135 | 7.89 | 0.5 | 6.97 | 37.46 | 28 | 7.43 | 17.65 | 30.02 | 265.2 | 12.04 | 2.87 | 40.98 | 1226.61 | 13.59 | 3 |
| 136 | 6.51 | 0.5 | 14.36 | 44.45 | 26.88 | 8.65 | 24.67 | 51.88 | 231.52 | 26.23 | 2.13 | 24.72 | 545.25 | 21.12 | 22 |
| 139 | 4.19 | 8.4 | 7.44 | 23.65 | 24.29 | 6.54 | 24.87 | 88.08 | 266.34 | 25.52 | 21 | 24.57 | 1419.92 | 16.7 | 4 |
| 152 | 3.31 | 0.5 | 1.47 | 14.87 | 3.08 | 5.51 | 14.32 | 30.11 | 203.68 | 7.49 | 0.58 | 40.51 | 1333.62 | 9.05 | 4 |
| 154 | 2.76 | 0.5 | 12.46 | 19.46 | 3.62 | 5.87 | 27.53 | 60.9 | 218.9 | 15.8 | 1.03 | 31.53 | 1547.01 | 19.08 | 3 |
| 155 | 5.58 | 0.5 | 3.28 | 20.45 | 23.71 | 5.9 | 18.54 | 33.46 | 187.94 | 3.15 | 1.51 | 20.64 | 1327.72 | 13.76 | 6 |
| 157 | 4.85 | 4.65 | 10.47 | 32.25 | 17.81 | 6.78 | 43.24 | 77.33 | 157.6 | 11.98 | 2.27 | 49.5 | 1577.5 | 28.88 | 6 |
| 168 | 3.31 | 0.5 | 12.65 | 47.54 | 19.46 | 4.42 | 12.77 | 28.46 | 208.5 | 17.39 | 0.55 | 37.88 | 1474.72 | 8.8 | 4 |
| 31 | 3.39 | 0.5 | 15.43 | 24.86 | 13.65 | 5.46 | 17.02 | 26.71 | 90.1 | 17.6 | 0.58 | 17.02 | 1616.69 | 11.57 | 4 |
| 47 | 2.47 | 0.5 | 12.4 | 43.06 | 5.54 | 4.52 | 14.56 | 34.82 | 259.07 | 14.98 | 0.89 | 31.93 | 1390.54 | 12.33 | 3 |
| 48 | 3.68 | 0.5 | 0.37 | 23.53 | 5.66 | 4.56 | 29.22 | 41.69 | 229.02 | 27.51 | 2.24 | 37.76 | 1413.33 | 13.59 | 6 |
| 132 | 2.67 | 6.87 | 14.32 | 37.89 | 15.78 | 5.54 | 49.47 | 88.54 | 228.5 | 37.55 | 6.35 | 52.88 | 1212.8 | 15.67 | 14 |
| 134 | 4.32 | 2.57 | 13.38 | 37.33 | 23.62 | 5.43 | 24.17 | 81.53 | 292.14 | 25.14 | 1.72 | 26.1 | 1009.3 | 21.81 | 7 |
| 156 | 7.71 | 0.5 | 15.17 | 33.02 | 21.05 | 6.65 | 28.8 | 46.47 | 226.5 | 20.59 | 4.67 | 47.4 | 1400.41 | 14.27 | 10 |

Blank indicates detection limit. [symbol]
indicates data missing or illegible when filed

### 2. Contents of Cytokine Panel Treatment Classification Test for Infantum Atopic Dermatitis

The contents required for cytokine panel test for unifying the evaluation with treatment of Th3 march of infantum atopic dermatitis (intervention of early medical treatment) are shown in Table 2.

TABLE 2
Cytokine Panel Treatment Classification Test for Infantum Atopic Dermatitis
Minimum Items

| | TGF | IL-4 | IL-5 | TL-6 | IL-9 | IL-13 | INF-? | TARC | IgE |
|---|---|---|---|---|---|---|---|---|---|
| Inhibitory | O | O | | | O | O | | | |
| factor | | | | | | | | | |
| Period of | O | *O | O | | | | O | | |
| Treatment | | | | | | | | | |

IL-6, IL-9/Th9 IL-4/Th2 TGF-β1/Th3 Znf/IL-13 IL5 IFN-?

### 3. Contents of Cytokine Panel Treatment Classification Examination for Severest Atopic Dermatitis

The contents required for cytokine panel test for unifying the evaluation with treatment of Th3 march of severest atopic dermatitis (intervention of early medical treatment) utilizing zinc cytokine signal transmission are shown in Table 3.

TABLE 3
Th Cell Signal Transmission System Cytokine Panel Test
(No Neuropathy) Minimum Items:
Treatment Classification Test for Severest Atopic Dermatitis

| | TGF-β1 | IL-4 | IL-5 | IL-6 | IL-9 | IL-13 | IFN-gamma |
|---|---|---|---|---|---|---|---|
| Inhibitory | O | | | | | | |
| factor | | | | | | | |
| Essence/ | O | O | O | | O | O | |
| Confusion | | | | | | | |
| Period | | | | | | | |
| Healing | O | | O | O | | | O |
| Period | | | | | | | |

This is a test for treatment, and shown the cases in which the cases the period was gone one and the cases confirmation test for evaluation were excluded.
IL-6, IL-9/Th9 IL-4/Th2 TGF-β1/Th3 Znf/IL-13 IL5 IFN-gamma

### 4. Contents of Cytokine Panel Treatment Classification Examination for Severest Atopic Dermatitis

The most typically clinical conditions of the severest atopic dermatitis in mammals utilizing zinc cell cytokine signal transmission are formed, and clinically, psychosis depending upon zing or Znt is exhibited. As long as integration of relation into Th3 march, the cure with the pharmaceutical composition of the present invention (“zinc finger Znf/PAD) (sometimes referred to as (zinc finger Znf) cannot be desired. So the contents required for cytokine panel test for unifying the evaluation with treatment of Th3 march dermatitis (early intervention of disease) were deliberated and are shown in Table 4. The details of the pharmaceutical composition of the present invention (“zinc finger Znf/PAD) are shown in Table 5 (hereinafter, similar is applied to the working examples).

TABLE 4

| | TGF-β1 | IL-4 | IL-5 | IL-6 | IL-9 | IL-13 | IFN-? | IP-10 | TNF |
|---|---|---|---|---|---|---|---|---|---|
| Incurable | O | | Oup | | | O | | | |
| factor | | | | | | | | | |
| Inhibitory | O | O | | | | | | | |
| factor | | | | | | | | | |
| Essence/ | | O | O | | | | | O | |
| Confusion | | | | | | | | | |
| Period | | | | | | | | | |
| Healing | O | | O | | O | | O | O | |
| Period | | | | | | | | | |
| Skin Barrier | | | | | | | O | | O |

| | | | Zn concentration | | |
|---|---|---|---|---|---|
| | TARC | Th3/TGF | in serum sZn | Ige | Bone-type ALP |
| Incurable | | O | | | |
| factor | | | | | |
| Inhibitory | O | | O | | |
| factor | | | | | |
| Essence/ | O | | | O down | O up |
| Confusion | | | | | |
| Period | | | | | |
| Healing | O | | | | |
| Period | | | | | |
| Skin | | | | | |
| Barrier | | | | | |

TABLE 5
<Detail of Zinc finger Znf/PAD>
Product Standard

| | | Standards/Interior | 360 g (200 mg/ |
|---|---|---|---|
| Name | Food | Content | 180 capsules) |
| Form | Capsule | Product Weight | 380 g |
| Form of | Silica gel Plastic | | |
| Package | Bottle | | |

| | |
|---|---|
| Raw Material | Zinc Yeast, Heated Pumpkin Seeds, Heated Corn Silk, |
| | Calcium, Phosphor (Mixture) |

Formulation of Materials

| | |
|---|---|
| Zinc Yeast | 30% |
| Heated Pumpkin Seeds | 20% |
| Heated Corn Silk | 20% |
| Calcium | 20% |
| Phosphor | 10% |

Form of Product
Size 2 Capsule Plastic Bottle 180 capsules
Production Stage
Collect Raw Materials/Seletion/Mixing/Filling/Packaging
Daily Adequate Intake 3 capsules
Change dosage depending upon body weight

### Example 3

### Cytokine Panel Test and Evaluation of Healing of Zinc Signal Th3 Atopic Dermatitis

(A kit containing zinc concentration in blood/delayed psychiatric disorder etc., Th9 allergic atopy cell/IL-9, BAP/BMP/TGF-β1 and for performing diagnosis and administration, The evaluation of healing by adding Znf activated/IL-13, see other Examples).

TABLE 6

[symbol]

| [symbol] | [symbol] | PDGF-bb | IL-1b | IL-1ra | IL-2 | IL-4 | IL-5 | IL6 | IL-7 | IL-8 | IL-9 | IL-10 | IL-12 | IL-13 |
|---|---|---|---|---|---|---|---|---|---|---|---|---|---|---|
| F | 22 | 164.02 | 0.73 | 93 64 | 8.97 | 0.76 | 2.45 | 4.2 | 2.31 | 9.52 | 1.72 | 0 19 | 2.89 | 2.95 |
| F | 40 | 1262.27 | 2.4 | 182.62 | 27.69 | 2.22 | 3.7 | 8 26 | 3.74 | 11.5 | 10 37 | 0 93 | 7.5 | 4.12 |
| M | 34 | 17.4 | 1.56 | 198.54 | 0 76 | 1.97 | 4.32 | 5.43 | 0.26 | 0.04 | 5 32 | 0.87 | 4.32 | 5.43 |
| F | 19 | 305.96 | 1.34 | 133.66 | 0.55 | 1.02 | 3.8 | 3.64 | 3.42 | 7.65 | 5 23 | 0 75 | 5.55 | 12.33 |
| F | 37 | 201.34 | 1.64 | 146.98 | 1.3 | 1.26 | 3.98 | 4.24 | 3.67 | 8.59 | 9 33 | 1.15 | 3.71 | 3.13 |
| F | 33 | 70.12 | 1.47 | 134.82 | 5 74 | 0 97 | 2.39 | 4.75 | 3.43 | 6.96 | 20.89 | 1 21 | 2.54 | 4 88 |
| F | 32 | 4837.21 | 2.67 | 324.5 | 12 31 | 1.98 | 5.97 | 7.99 | 3.69 | 13.1 | 13 28 | 1.91 | 12 68 | 4 12 |
| F | 21 | 716.66 | 1.92 | 210.96 | 8 02 | 1.62 | 4.3 | 7.07 | 4.95 | 10.4 | 7.86 | 1 68 | 11 48 | 6.27 |
| [symbol] | IL-15 | IL-17 | Eotaxin | FGF | G-CSF | M-CSF | IFN-g | IP-10 | (MCAF) | MIP-1a | MIP-1b | ANTES | TNF-a | VEGF |
| F | 0.5 | 0.1 | 25.56 | 34.21 | 4.06 | 5 59 | 23.16 | 220.7 | 13 1 | 2.67 | 36 23 | 1559 6 | 5.45 | 2.76 |
| F | 0.5 | 7.12 | 40 57 | 22.27 | 15.59 | 51.55 | 83.86 | 224 1 | 20.64 | 1.59 | 31.42 | 1204.6 | 23 | 2.92 |
| M | 0.5 | 0.1 | 4.32 | 17.65 | 2.45 | 4.32 | 43.67 | 36.05 | 16 54 | 1.65 | 32.16 | 37.45 | 26.54 | 4.32 |
| F | 0.5 | 3.07 | 33.02 | 9 25 | 5 46 | 16 94 | 37.86 | 214 3 | 12.65 | 1.33 | 31.57 | 1267 3 | 12.07 | 1.86 |
| F | 0.5 | 6.76 | 32 09 | 17.81 | 7.28 | 20.92 | 41.36 | 221 9 | 20.24 | 0.75 | 27 3 | 1372 | 17.99 | 1.52 |
| F | 0.5 | 0.5 | 23 91 | 13 28 | 6 02 | 16.69 | 39.53 | 320.2 | 15.37 | 1.06 | 21 33 | 1663 6 | 14.27 | 0 02 |
| F | 4.37 | 54.4 | 12.74 | 42 68 | 9.53 | 39.97 | 77.63 | 310.2 | 10 7 | 4 93 | 69.81 | 766 24 | 27 51 | 1 78 |
| F | 0.5 | 31.7 | 26.34 | 50.85 | 6.11 | 38.25 | 51.01 | 250 2 | 15 04 | 4 15 | 42.24 | 1294 8 | 20.7 | 9 96 |

[symbol]

indicates data missing or illegible when filed

A frozen blood plasma of mild atopic dermatitis of 9 subjects was used as a sample.

Results: The contents of cytokine panel treatment classification examination and the contents for using a kid for diagnosis and treatment utilizing a novel zinc finger Znf/PAD are shown in Table 6.

The contents concerned are shown by bolded mark. The cytokine panel contents to be incorporated into the minimum panel are IL-1b, IL-1ra, IL-2, IL-4, IL-5, IL-17, IL-13 and IL-9 (evaluation of healing) and FGF, G-CSF, G-CSF, IP-10 and the like. In this example, the measurements were made including other items such as RANTES and VEGE (items relating to pollinosis) and 27 kinds of cytokine panels.

### Example 4

### Cytokine Panel Test and Evaluation of Healing of Th3 March of Pollinosis, Th3 March or Pollinosis

(A kit containing zinc concentration in blood/delayed psychiatric disorder etc., Th9 allergic atopy cell/IL-9, BAP/BMP/TGF-β1 and for performing diagnosis and administration. The evaluation of healing by adding Znf activated/IL-13).

TABLE 7
Cytokine Panel of Pollenosis for Utilizing Th3 March

| Sex | Age | PDGF | Il-1b | IL-1ra | IL-2 | IL-4 | IL-5 | IL-6 | IL-7 | IL-8 | IL-9 | IL-10 | IL-12 | L13 |
|---|---|---|---|---|---|---|---|---|---|---|---|---|---|---|
| F | 42 | 624.14 | 2.08 | 212.16 | 5.56 | 1.68 | 5.31 | 6.07 | 5.22 | 11.51 | 9.75 | 1.75 | 20.29 | 4.94 |
| F | 45 | 5717.74 | 252 | 319.03 | 11.09 | 2.13 | 4.49 | 8.47 | 4.76 | 13.74 | 18.71 | 2.24 | 21.26 | 9.19 |
| F | 36 | 612.16 | 237 | 278.1 | 13.81 | 1.82 | 5.96 | 8.17 | 4.48 | 13.93 | 15.39 | 1.58 | 10.54 | 5.93 |
| F | 34 | 1771.4 | 123 | 112.4 | 4.77 | 0.78 | 3.24 | 3.54 | 7.61 | 7.75 | 5.77 | 0.4 | 7.55 | 6.58 |
| F | 33 | 672.01 | 119 | 146.96 | 1.81 | 1.26 | 3.68 | 4.08 | 3.02 | 8.38 | 9.41 | 0.47 | 9.56 | 4.61 |
| M | 33 | 459.45 | 0.56 | 75.64 | 1.76 | 0.58 | 19 | 3.18 | 17.7 | 6.08 | 8.86 | 0.43 | 7.54 | 1.99 |
| F | 41 | 638.47 | 1.61 | 167.96 | 2.39 | 1.49 | 3.73 | 4.49 | 32.3 | 8.89 | 7.62 | 1.03 | 7.76 | 5.58 |
| F | 32 | 956.2 | 3.48 | 460.85 | 26.49 | 3.14 | 9.25 | 12.56 | 8.42 | 20.89 | 24.26 | 3.82 | 23.53 | 9.31 |
| F | 32 | 3287.11 | 1.81 | 200.9 | 4.77 | 1.89 | 5.01 | 5.23 | 12.16 | 10.47 | 8.81 | 1.53 | 17.08 | 5.76 |
| F | 43 | 704.78 | 0.7 | 107.84 | 0.26 | 0.83 | 2 | 4.04 | 2.88 | 5.76 | 10.28 | 0.16 | 2.45 | 3.41 |
| F | 34 | 522.3 | 0.49 | 45.32 | 3.11 | 0.73 | 1.76 | 2.63 | 1.18 | 5.85 | 3586.44 | 0.47 | 6.63 | 9.03 |
| F | 42 | 397.1 | 1.43 | 112.06 | 8.51 | 1.34 | 4.13 | 4.13 | 3.24 | 10.75 | 7.11 | 1.41 | 12.87 | 8.77 |
| F | 27 | 508.01 | 1.35 | 149.3 | 1.77 | 1.03 | 3.56 | 3.96 | 3.58 | 8.45 | 7.13 | 1.08 | 5.17 | 4.06 |
| F | 51 | 234.62 | 1.45 | 138.29 | 10.67 | 0.97 | 4.86 | 7.67 | 3.52 | 10.84 | 25.11 | 0.96 | 8.01 | 27.43 |
| F | 49 | 502.34 | 2.16 | 193.81 | 7.61 | 1.82 | 5.06 | 6.54 | 4.17 | 11.98 | 19.96 | 2.18 | 13.09 | 9.22 |
| F | 34 | 234.98 | 0.6 | 103.28 | 7.05 | 0.8 | 1.71 | 4.73 | 4.79 | 6.45 | 4.68 | 0.71 | 4.33 | 2.25 |
| F | 45 | 196.56 | 2.12 | 199.72 | 5.75 | 1.66 | 4.92 | 5.93 | 4.09 | 13.98 | 12.59 | 1.13 | 9.07 | 12.91 |
| F | 41 | 314.83 | 1.97 | 179.69 | 25.83 | 1.6 | 4.5 | 6.34 | 5.61 | 13.69 | 14.47 | 2.88 | 12.52 | 6.32 |
| F | 40 | 199.43 | 1.07 | 82.93 | 5.46 | 1.29 | 2.29 | 3.51 | 3.08 | 7.82 | 5 | 0.64 | 3.57 | 3.81 |
| F | 22 | 1121.85 | 0.88 | 123.87 | 4.32 | 1.04 | 2.91 | 3.16 | 5.87 | 7.14 | 4.1 | 0.57 | 6.55 | 3.77 |
| F | 39 | 1175.53 | 1.94 | 213.33 | 5.67 | 1.82 | 3.81 | 5.4 | 4.14 | 11.6 | 10.39 | 0.66 | 8.5 | 4.88 |
| M | 23 | 195.06 | 1.01 | 99.87 | 4.37 | 0.74 | 2.52 | 3.06 | 1.77 | 9.45 | 8.48 | 0.41 | 7.89 | 5.64 |
| M | 42 | 187.28 | 1.06 | 108.41 | 0.43 | 0.94 | 3.94 | 4.17 | 2.94 | 6.68 | 18.08 | 0.61 | 4.57 | 17.07 |
| M | 59 | 365.49 | 2.15 | 183.21 | 10.53 | 2.18 | 5.93 | 6.63 | 4.95 | 14.99 | 81.15 | 2.54 | 13.79 | 13.91 |
| M | 42 | 252.29 | 0.96 | 87.93 | 1.06 | 1 | 2.74 | 4.14 | 3.47 | 8.07 | 13.39 | 1.36 | 5.58 | 15.3 |
| M | 48 | 2137.08 | 2.68 | 288.16 | 13.32 | 1.76 | 6.43 | 7.87 | 3.56 | 13.55 | 19.87 | 1.9 | 15.89 | 20.46 |
| M | 37 | 176.43 | 0.87 | 95.07 | 0.65 | 0.78 | 2.02 | 3.64 | 2.42 | 6.08 | 7.73 | 0.52 | 4.82 | 3.22 |
| F | 22 | 164.07 | 0.73 | 93.64 | 0.54 | 0.76 | 2.48 | 4.7 | 2.31 | 9.57 | 1.72 | 0.19 | 2.89 | 2.95 |
| M | 29 | 1521.64 | 1.3 | 162.12 | 2.05 | 1.31 | 3.52 | 4.45 | 2.51 | 8.26 | 9.71 | 1.16 | 10.07 | 4.76 |
| M | 43 | 1100.2 | 1.49 | 180.74 | 0.52 | 1.26 | 3.67 | 5.26 | 3.55 | 10.32 | 9.3 | 0.72 | 10.54 | 6.42 |
| F | 43 | 3317 | 0.54 | 52.95 | 1.84 | 0.71 | 1.75 | 3.39 | 1.61 | 7.1 | 3.89 | 0.51 | 1.88 | 1.64 |
| F | 43 | 37102 | 1.08 | 101.01 | 7.01 | 0.89 | 2.42 | 6.06 | 2.04 | 8.45 | 4.44 | 0.44 | 5.41 | 7.66 |
| F | 26 | 195.18 | 0.74 | 118.13 | 4.37 | 0.79 | 2.7 | 3.17 | 1.84 | 6.82 | 4.06 | 0.6 | 2.86 | 4.39 |
| M | 29 | 160.99 | 0.8 | 75.08 | 5.43 | 0.94 | 2.41 | 2.96 | 2.65 | 5.94 | 2.54 | 0.54 | 3.62 | 4.75 |
| F | 28 | 488.97 | 1.49 | 96.47 | 8.37 | 1.14 | 2.97 | 16.65 | 2.65 | 7.56 | 9.74 | 1.17 | 9.53 | 3.68 |
| M | 27 | 288.9 | 1.76 | 191.46 | 6.98 | 1.53 | 4.18 | 6.7 | 2.68 | 9.55 | 23.92 | 1.53 | 6.34 | 7.89 |
| F | 28 | 651.33 | 1.19 | 145.82 | 2.01 | 1.17 | 3.42 | 4.95 | 7.42 | 8.17 | 7.54 | 0.6 | 11.65 | 3.99 |
| F | 25 | 165.37 | 1.88 | 119.28 | 58.77 | 1.21 | 2.79 | 9.44 | 3.56 | 9.66 | 2.48 | 0.23 | 7.26 | 3.06 |
| F | 39 | 463.74 | 1.1 | 81.8 | 0.55 | 1.16 | 1.98 | 3.21 | 1.95 | 6.36 | 3.51 | 0.1 | 1.12 | 1.35 |
| F | 46 | 469.46 | 0.8 | 94.2 | 6.43 | 0.76 | 2.02 | 5.63 | 1.8 | 5.69 | 3.63 | 0.64 | 10.18 | 5.32 |
| M | 53 | 146.77 | 2.7 | 209.78 | 5.04 | 1.66 | 19.8 | 6.85 | 5.03 | 16.28 | 11.13 | 1.03 | 6.74 | 9.84 |
| F | 54 | 390.38 | 1.54 | 160.37 | 3.94 | 1.43 | 3.23 | 4.6 | 7.88 | 7.75 | 8.07 | 1.23 | 6.69 | 8.13 |
| M | 34 | 174 | 1.65 | 165.4 | 4.32 | 1.43 | 4.22 | 3.71 | 0.26 | 0.04 | 4.32 | 1.54 | 5.43 | 3.54 |
| F | 31 | 50.36 | 105 | 115.76 | 5.43 | 0.95 | 2.54 | 3.33 | 2.56 | 6.73 | 1.54 | 0.67 | 1.69 | 1.12 |
| M | 40 | 227.83 | 2.7 | 270.88 | 10.28 | 1.65 | 6.4 | 7.6 | 5.37 | 15.8 | 22.53 | 2.41 | 12.22 | 22.86 |
| F | 42 | 966.11 | 1.97 | 212.76 | 4.73 | 1.57 | 4.84 | 4.78 | 4.17 | 10.84 | 7.32 | 1.56 | 12.46 | 5.03 |
| F | 31 | 399.48 | 2.17 | 209.74 | 9.41 | 1.65 | 5.17 | 7.32 | 4.13 | 11.41 | 9.85 | 1.86 | 11.11 | 6.9 |
| F | 41 | 289.35 | 1.95 | 166.21 | 15.27 | 1.78 | 3.22 | 5.36 | 3.06 | 9.43 | 7.05 | 1.39 | 6.66 | 4.91 |
| F | 32 | 165.29 | 1.7 | 169.72 | 6.71 | 1.5 | 4.8 | 5.6 | 4.76 | 10.51 | 10.45 | 1.14 | 3.76 | 3.65 |
| F | 50 | 455.46 | 1.5 | 115.26 | 3.94 | 1.7 | 3.34 | 5.08 | 7.94 | 7.75 | 6.19 | 0.81 | 3.98 | 2.81 |
| F | 58 | 332.47 | 2.62 | 250.88 | 25.23 | 1.97 | 6.18 | 8.05 | 5.77 | 14.36 | 17.12 | 1.13 | 15.99 | 10.74 |
| M | 28 | 370.08 | 1.51 | 122.15 | 0.18 | 1.07 | 3.21 | 4.66 | 3.63 | 8.31 | 3.89 | 0.54 | 3.92 | 2.58 |
| F | 19 | 305.96 | 1.34 | 133.66 | 0.55 | 1.02 | 2.8 | 3.64 | 3.42 | 7.65 | 5.23 | 0.75 | 5.55 | 12.33 |
| F | 37 | 201.34 | 1.64 | 146.95 | 1.3 | 1.26 | 3.48 | 4.74 | 3.67 | 8.59 | 9.33 | 1.15 | 3.71 | 3.13 |
| F | 28 | 1605.75 | 2.12 | 138.29 | 19.79 | 1.66 | 3.02 | 7.86 | 2.96 | 13.46 | 7.11 | 0.71 | 7.07 | 2.4 |
| F | 27 | 2434.4 | 1.58 | 111.83 | 3.21 | 0.95 | 2.69 | 3.6 | 2.99 | 8.31 | 3.6 | 0.57 | 5.44 | 2.78 |
| F | 32 | 4837.21 | 2.67 | 324.5 | 17.31 | 1.98 | 5.97 | 7.95 | 3.69 | 13.17 | 13.28 | 1.91 | 12.68 | 4.12 |
| F | 47 | 1006.1 | 3.06 | 137.51 | 8.86 | 1.99 | 5.32 | 5.64 | 4.47 | 11.6 | 11.46 | 0.97 | 2.84 | 2.81 |

| Sex | Age | IL-15 | IL-17 | Eotaxin | FGF | G-CSF | GM-SCF1 | IFN-? | IP-10 | MCP-11 | MIP-1a | MIP-1b | ANTES | TNF-a | VEGF |
|---|---|---|---|---|---|---|---|---|---|---|---|---|---|---|---|
| F | 42 | 39 | 16.64 | 42.84 | 29.75 | 12.84 | 36.7 | 60.12 | 216.87 | 20.71 | 7.69 | 40.44 | 1254.16 | 70.11 | 26 |
| F | 45 | 69.3 | 41.07 | 64.64 | 49.99 | 13.23 | 44.27 | 84.77 | 378.96 | 19.05 | 3.9 | 46.57 | 999.04 | 32.46 | 22.0 |
| F | 36 | 5.68 | 14.57 | 41.01 | 18.19 | 17.6 | 37.99 | 75.03 | 473.58 | 10.56 | 2.4 | 23.98 | 1141.77 | 26.49 | 19 |
| F | 34 | 0.5 | 6.91 | 20.98 | 16.88 | 21.1 | 17.77 | 38.03 | 171.45 | 6.34 | 2.34 | 24.23 | 1319.75 | 35.54 | 5.4 |
| F | 33 | 0.5 | 8.34 | 35.08 | 33.95 | 33.71 | 24.59 | 43.68 | 179.38 | 15.3 | 1.9 | 22.94 | 1411.62 | 9.72 | 7.7 |
| M | 33 | 0.5 | 0.1 | 36.04 | 15.02 | 5.22 | 11.64 | 20.08 | 267.92 | 25.35 | 0.9 | 31.94 | 965.65 | 2.72 | 0.7 |
| F | 41 | 0.5 | 12.3 | 48.94 | 20.8 | 7.6 | 24.76 | 46.63 | 271.67 | 13.92 | 1.69 | 24.71 | 1416.02 | 14.18 | 6.9 |
| F | 32 | 12.89 | 35.22 | 40.86 | 67.88 | 25.36 | 60.28 | 114.02 | 202.74 | 14.9 | 5.75 | 30.1 | 1093.78 | 52.26 | 8.3 |
| F | 32 | 0.83 | 19.66 | 35.63 | 35.63 | 6.6 | 32.19 | 60.42 | 255.66 | 9.19 | 3.88 | 30.63 | 1286.17 | 13.76 | 21.0 |
| F | 43 | 0.5 | 9.35 | 18.65 | 18.55 | 6.92 | 14.74 | 28.28 | 264.86 | 18.95 | 1.8 | 50.49 | 1403.46 | 13.47 | 1.32 |
| F | 34 | 0.5 | 1.6 | 36.84 | 73.71 | 6.92 | 33.98 | 12.18 | 385.54 | 19.58 | 3.11 | 31.76 | 1433.1 | 3.05 | 29 |
| F | 42 | 0.5 | 10.96 | 42.1 | 18.09 | 7.36 | 15.96 | 47.69 | 505.81 | 7.11 | 1.54 | 25.15 | 1405.47 | 15.79 | 16.62 |
| F | 27 | 0.5 | 7.76 | 20.85 | 13.59 | 8.04 | 17.68 | 39.53 | 303.18 | 9.67 | 18.9 | 25.15 | 1453.77 | 11.99 | 4.32 |
| F | 51 | 6.35 | 8.5 | 77.85 | 19.01 | 9.35 | 27.45 | 49.8 | 418.17 | 24.77 | 2.4 | 35.1 | 1676.03 | 22.74 | 6.94 |
| F | 49 | 7.32 | 28.96 | 50.99 | 40.75 | 13.26 | 29.9 | 59.79 | 350.85 | 21.5 | 3.48 | 43.49 | 1800.75 | 31.61 | 20.06 |
| F | 34 | 2.63 | 0.54 | 68.98 | 37.54 | 7.45 | 10.03 | 1.75 | 1090.6 | 31.594 | 4.37 | 32.58 | 1296.74 | 6.79 | 3.02 |
| F | 45 | 4.82 | 12.79 | 42.94 | 17.72 | 8.14 | 25.76 | 55.34 | 229.52 | 11.47 | 7.44 | 21.89 | 1279.74 | 18.24 | 4.04 |
| F | 41 | 14.24 | 27.41 | 65.79 | 35.55 | 313.8 | 31.94 | 67.31 | 363.23 | 40.32 | 4.5 | 50.13 | 1413.33 | 23.93 | 13.51 |
| F | 40 | 0.5 | 2.01 | 42.71 | 7.35 | 2.55 | 18.18 | 33.63 | 225.64 | 11.5 | 3.71 | 29.53 | 1544.39 | 6.54 | 6.43 |
| F | 22 | 0.5 | 4.82 | 31.45 | 2.87 | 6.46 | 10.36 | 33.63 | 271.84 | 14.75 | 0.82 | 19.06 | 1496.79 | 7.04 | 8.58 |
| F | 39 | 5.87 | 18.43 | 31.13 | 20.89 | 18.1 | 26.1 | 59.16 | 404.22 | 17.77 | 2.18 | 35.75 | 1651.49 | 15.11 | 6.37 |
| M | 23 | 0.5 | 3.89 | 70.94 | 32.14 | 6.17 | 12.77 | 24.59 | 299.75 | 16.74 | 2.56 | 40.66 | 1328.36 | 6.7 | 5.43 |
| M | 42 | 0.5 | 7.65 | 25.41 | 11.33 | 7.65 | 16.53 | 33.12 | 317.55 | 19.41 | 1.81 | 30.19 | 1521.17 | 19.68 | 8.28 |
| M | 59 | 11.63 | 18.39 | 43.61 | 26.95 | 14.4 | 54.16 | 60.8 | 372.17 | 24.79 | 3.6 | 45.85 | 1750.48 | 28.07 | 18.69 |
| M | 42 | 0.5 | 0.59 | 72.67 | 2.69 | 6.6 | 25.6 | 37.02 | 353.02 | 14.96 | 12 | 35.7 | 1530.37 | 8.04 | 2.26 |
| M | 48 | 10.63 | 45.87 | 46.43 | 51.35 | 10.2 | 47.91 | 80.68 | 217.8 | 11.04 | 4.79 | 27.93 | 1717.44 | 79.39 | 10.99 |
| M | 37 | 0.5 | 9.46 | 43.47 | 18.55 | 5.77 | 13.76 | 25.83 | 221.94 | 17.74 | 1.42 | 66.51 | 1885.5 | 11.99 | 0.19 |
| F | 22 | 0.5 | 0.1 | 26.56 | 16.54 | 4.06 | 5.59 | 23.16 | 220.69 | 13.1 | 1.65 | 36.23 | 1559.59 | 5.45 | 0.56 |
| M | 29 | 1.98 | 1.47 | 41.57 | 23.12 | 7.06 | 27.2 | 43.02 | 161.74 | 17.13 | 2.03 | 77.85 | 1149.99 | 16.46 | 5.04 |
| M | 43 | 2.57 | 1.47 | 45.15 | 15.07 | 6.77 | 25.54 | 48.1 | 385.67 | 18.63 | 7.06 | 57.17 | 1468.71 | 15.11 | 11.7 |
| F | 43 | 2.34 | 0.1 | 163.31 | 21.54 | 4.38 | 7.96 | 23.16 | 348.27 | 324.34 | 0.11 | 33.15 | 1598.05 | 5.04 | 8.16 |
| F | 43 | 0.5 | 1.47 | 72.73 | 37.1 | 5.73 | 16.94 | 26.71 | 881.06 | 17.46 | 0.54 | 34.75 | 1304.04 | 3.54 | 1.07 |
| F | 26 | 0.5 | 1.47 | 11.34 | 0.79 | 5.32 | 15.05 | 33.63 | 227.96 | 21.66 | 0.57 | 26.12 | 1278.05 | 9.72 | 9.65 |
| M | 29 | 0.5 | 0.1 | 42.11 | 23.54 | 5.17 | 11.56 | 16.95 | 195.57 | 21.16 | 0.18 | 18.41 | 1491.22 | 5.2 | 0.65 |
| F | 38 | 0.91 | 6.7 | 31.98 | 12.75 | 6.77 | 27.87 | 45.65 | 702.76 | 23.13 | 1.69 | 37.19 | 1971.68 | 7.79 | 0.15 |
| M | 27 | 0.5 | 11.92 | 28.42 | 20.09 | 9.49 | 32.11 | 54.07 | 628.26 | 18 | 2.91 | 28.86 | 1439.99 | 16.63 | 4.92 |
| F | 38 | 0.5 | 11.14 | 34.13 | 21.5 | 6.94 | 15.71 | 36.34 | 287.95 | 12.43 | 1.65 | 42.37 | 1483.23 | 10.05 | 11.96 |
| F | 25 | 18 | 0.1 | 114.54 | 32.1 | 19.5 | 38.75 | 37.86 | 265.06 | 20.94 | 2.84 | 22.64 | 1788.69 | 8.71 | 0.56 |
| F | 39 | 0.5 | 0.1 | 38.89 | 37.65 | 11.43 | 17.93 | 33.8 | 210.69 | 20.72 | 0.5 | 27.64 | 1402.07 | 6.7 | 0.87 |
| F | 46 | 3.78 | 5.76 | 67.03 | 75.19 | 7.51 | 20.17 | 27.41 | 119.82 | 6.97 | 2.44 | 14.3 | 1169 | 4.37 | 432 |
| M | 53 | 4.07 | 6.18 | 72.26 | 21.15 | 14.82 | 35.94 | 59.63 | 143.04 | 29.33 | 1..89 | 75.83 | 1126.85 | 79.05 | 4.18 |
| F | 54 | 0.5 | 5.13 | 44.76 | 7.48 | 7.23 | 76.02 | 38.53 | 347.92 | 22.06 | 1 | 45.67 | 1119.55 | 16.46 | 6.99 |
| M | 34 | 0.5 | 0.1 | 44.63 | 4.32 | 3.31 | 12.54 | 12.65 | 36.05 | 17.65 | 0.54 | 23.65 | 3745 | 14.65 | 17.65 |
| F | 31 | 0.5 | 0.1 | 21.59 | 55 | 5.36 | 10.59 | 24.76 | 216.15 | 12.35 | 0.46 | 25.54 | 694.84 | 9.89 | 6.98 |
| M | 40 | 9.9 | 21.49 | 59.61 | 44 | 14.02 | 30.66 | 67.78 | 198.75 | 24.01 | 2.61 | 21.83 | 1337.68 | 31.85 | 6.89 |
| F | 42 | 0.5 | 11.49 | 27.31 | 24.62 | 13.32 | 28.04 | 56.94 | 233.48 | 17.18 | 1.87 | 26.14 | 732.79 | 12.92 | 19.16 |
| F | 31 | 0.5 | 24.24 | 33.12 | 33.26 | 8.41 | 26.19 | 53.42 | 277.04 | 15.66 | 2.69 | 47.67 | 1601.39 | 18.33 | 14.71 |
| F | 41 | 0.5 | 21.31 | 80.88 | 28.83 | 8.48 | 39.37 | 40.2 | 197.57 | 23.78 | 1.76 | 28.8 | 1537.42 | 21.21 | 7.73 |
| F | 33 | 0.5 | 5.97 | 75.64 | 18.09 | 6.16 | 22.5 | 44.5 | 285.54 | 16.88 | 0.88 | 33.62 | 1275.13 | 22.23 | 0.57 |
| F | 50 | 0.5 | 6.07 | 32.39 | 18.74 | 7.09 | 25.35 | 37.09 | 298.01 | 29.11 | 1.47 | 26.72 | 1526.02 | 15.96 | 0.62 |
| F | 68 | 10.82 | 28.28 | 58.9 | 56.78 | 11.21 | 58.61 | 64.04 | 285.7 | 18.23 | 5.46 | 37.34 | 978.86 | 78.7 | 7.09 |
| M | 28 | 0.5 | 7.97 | 39.61 | 13.17 | 6.16 | 17.35 | 45.48 | 298.94 | 21.08 | 0.68 | 76.37 | 1435.79 | 13 | 0.62 |
| F | 19 | 0.5 | 3.07 | 33.07 | 9.25 | 5.46 | 16.94 | 37.86 | 214.31 | 12.65 | 1.33 | 31.57 | 1767.27 | 12.07 | 1.86 |
| F | 37 | 0.5 | 6.76 | 37.9 | 17.81 | 7.28 | 20.92 | 41.36 | 221.94 | 20.24 | 0.75 | 27.3 | 1371.95 | 17.59 | 1.62 |
| F | 28 | 12.1 | 11.33 | 34.09 | 16.68 | 8.11 | 31.09 | 74.57 | 133.11 | 15.73 | 1.37 | 33.23 | 1257.55 | 19.18 | 0.47 |
| F | 27 | 0.5 | 4 | 21.15 | 16.1 | 8.7 | 24.84 | 33.8 | 361.23 | 12.87 | 1.91 | 30.51 | 1359.13 | 9.3 | 0.73 |
| F | 32 | 4.37 | 54.42 | 12.74 | 47.68 | 9.53 | 39.97 | 77.63 | 310.17 | 10.7 | 4.93 | 69.81 | 766.24 | 27.51 | 7.54 |
| F | 47 | 0.5 | 0.1 | 38.28 | 9.96 | 8.16 | 30.41 | 77.33 | 244.3 | 23.55 | 1.28 | 27.05 | 1329.78 | 22.08 | 5.43 |

A frozen blood plasma of mild atopic dermatitis of 58 subjects was used as a sample.

Results: The contents of cytokine panel treatment classification examination (performed for new definition) and the contents for using a kid for diagnosis and treatment utilizing a novel zinc finger Znf/PAD are shown in the table.

The contents concerned are shown by marked (red). The cytokine panel contents to be incorporated into the minimum panel are IL-1b, IL-1ra, IL2, IL-4, IL-5, IL-6, IL-7, IL-9, IL-10, IL-12, IL-13, IL-15, IL-17, Eotaxin, FGF, G-CSF, G-CSF, INF-γ, IP-10, MIP-1b, TNF-α and the like (different from the standard cytokine panel). In the intervention of early medical treatment, Th1 vital reaction, interferon-γ, IL-13, and IL-9 etc. are included in the cytokine panel as the evaluation of the treatment, and the diagnosis and treatment are performed all at once. In this example, the measurements were made including other items.

Example of Patient: Male Patient in late 40s Affected with Pollinosis

TABLE 8

| Deduced Items | Standard value | | Pollinosis |
|---|---|---|---|
| Blood Collection | (Simultaneous | | 2010.3.02 |
| Date | Measurement) | | |
| TFG-β1/Th3 | 1.1-5 | ng/ml or more | 0.8 |
| PDGF-bb | | | 207.36 |
| IL-1b | 1 | pg/ml or more | 0.37 |
| IL-1ra | | | 44.37 |
| IL-2 | 10 | pg/ml or more | 5.9 |
| IL-4/Th2 | 2 | pg/ml or more | 0.39 |
| IL-5 | 3 | pg/ml or more | 0.65 |
| IL-6 | 10 | pg/ml or more | 1.85 |
| IL-7 | | | 0.63 |
| IL-8 | 100 | pg/ml or more | 3.35 |
| IL-9/Th9 | 30 | pg/ml or more | 10.76 |
| IL-10 | 1.0 | pg/ml or more | 2.21 |
| IL-12 | 20 | pg/ml or more | 4.05 |
| IL-13 (Znf) | 2 | pg/ml or more | 1.15 |
| IL-15 | 10.0 | pg/ml or more | 3.04 |
| IL-17/Th17 | 20 | pg/ml or more | 2.8 |
| Eoxtaxin | | | 2.3 |
| FGF | 50 | pg/ml or more | 0 |
| G-SCF | | | 6.7 |
| GM-CSF | 50.0 | pg/ml or more | 11.97 |
| IFN-? | 100 | pg/ml or more | 18.4 |
| IP-10 | | | 130.67 |
| MCP-1 (MCA) | | | 34.96 |
| MIP-1a | | | *0.52 |
| MIP-1b | | | 52.78 |
| RANTES | 2000 | pg/ml or more | 884.04 |
| TNF- | 30 | pg/ml or more | 13.34 |
| VEGF (45) | | | 20.02 |

| | | |
|---|---|---|
| IFN-g/IL-4 | Characteristic Absent | Not Calculated |

Results: Availability of the cytokine panel has been confirmed, making it possible to use the kit for intervention of early medical treatment utilizing Th3 march. It was decided from the cytokine panel that no IL-4 was produced, there was strong vascular disorder (VEGF and PANTES), there was abnormality of TGF/FGF, and Th3 march was at a control period. The patient was under the clinical condition where cartilage of respiratory system and connective tissues were weaken and vascular permeability was emphasized, the panel was proven to be treatment classification examination in which exhibition of the symptom (nasal discharge etc.) by stimulation a (pollen dusts) can be well understood.

### Example 5

### Cytokine Panel Test of Dog Th2-Th3 Shift March EDS and Evaluation of Healing and DNA Repair Capacity with the Pharmaceutical Composition of the Present Invention

The results of confirmation of the cytokine panel test of dog EDS which is Znt gene defect disease are shown in the following Table 9.

TABLE 9

| Items | |
|---|---|
| TGF-β1 | 9.0 ng/ml |
| IL-1b | 0.42 |
| IL-1ra | *4.43 |
| IL-2 | 0 |
| IL-4/Th2 | 0 |
| IL-5 | 0 |
| IL-6 | 0 |
| IL-7 | 0.23 |
| IL-8 | *0.94 |
| IL-9/Th9 | *1.06 |
| IL-10 | 0 |
| IL-12 | 0 |
| IL-13/Znf | 0 |
| IL-15 | 0 |
| IL-17 | 0 |
| Eoxtaxin | 0 |
| FGF | *0.22 |
| G-SCF | 0 |
| GM-CSF | 0 |
| IFN- | 0 |
| IP-10 (48) | 0 |
| MCP-1 (MCA) | 3.79 |
| MIP-1a (55) | 0 |
| MIP-1b (18) | 0 |
| RANTES | 4.97 |
| TNF-a (36) | 0 |
| VEGF (45) | 0 |
| IFN-g/IL-4 | #Value |

Results: It has been observed to be typical Th3 march disease and to be strong allergy. The disease was treated with the pharmaceutical composition of the present invention and improvement of hair growth and that of fur were observed.

This was a dog's case of FDS influence upon Znt or Znf and FDS was a low value; the evaluation could be made by EDS and cytokine panel including the clinical diagnosis.

### Example 6

### Cytokine Panel Test of EDS as Dog Th23 March Related Disease and Evaluation of Healing and DNA Repair Capacity with the Pharmaceutical Composition of the Present Invention

The results of confirmation of the cytokine panel test of dog EDS which is Znt gene defect disease are shown in the following Table 9.

TABLE 10

| Items | EDS (Dog) |
|---|---|
| Blood Collected @ | 2010.3.26 |
| TGF-β1/Th3 | 7.1 |
| PDGF-bb | 411.84 |
| IL-1b | 0.35 |
| IL-1ra | 2907.6 |
| IL-2 | 0.66 |
| IL-4/Th2 | 0.41 |
| IL-5 | 0.48 |
| IL-6 | 0.91 |
| IL-7 | 0.59 |
| IL-8 | 1.57 |
| IL-9/Th9 | 2.47 |
| IL-10 | 0.36 |
| IL-12 | 1.03 |
| IL-13/Znf | 0.73 |
| IL-15 | 0 |
| IL-17/Th17 | 0 |
| Eoxtaxin | 0 |
| FGF | 15.6 |
| G-SCF | 2 |
| GM-CSF | 7.5 |
| IFN-? | 54.83 |
| IP-10 | *0.33 |
| MCP-1 (MCA) | 0 |
| MIP-1a | 1.48 |
| MIP-1b | 1.35 |
| RANTES | 7.54 |
| TNF- | 0 |
| VEGF (45) | 0 |
| IFN-g/IL-4 | Not Calculated |

Results: It was differentially diagnosed to be atopy. As pathology, see other examples.

Although the zinc concentration in blood serum was low, the clinical condition was improved. It could be seen that the characteristics of high Th9 cell and conversely of low IP-10, and the example was observed to meet Th3 march (see other examples).

(Amongst Th3 march, Th1-Th2 march is shown by other examples. Inhibitory factor of treatment of atopy.)

### Example 7

### Effectiveness of Cytokine Panel Against Atopic Dermatitis

In atopic dermatitis, Th9/IL-9 was more effective rather than Th2 as a bio-parameter.

Th9/IL-9 is so-called allergy responsible cell and produces IL-9 more than 10 times IL-4, decreases in conjugation with the improvement of mammal atopic allergy, and particularly, inverse correlation can be seen at the time of treating atopy. IL-9/Th9 cell was proven to be a novel parameter for treatment of atopy. In the case of severest atopy patient, when TGF-β1 in the blood serum was high and when Th3 vital reaction (in vivo dynamics of Th cell at the time of administration of a drug) and Th1 vital reaction were low, IL-9/Th9 was regulated while IL-13 (Znf activity) was increased in the case where the therapeutic effect could be seen. At the time of healing mammal atopy, similar phenomena of IL-9/Th9 cell and IL-13 (Znf activity) could be seen, making it clear that the main loci of the treatment are IL-9 and IL-13 for the first time.

### (Healing Formula and Evaluation by Cytokine Panel in Severest Atopy Patient)

The results of the cytokine panel test in clinically improved treatment examples are shown (FIG. 5).

IL-4/Th2 cell which is a factor of atopic dermatitis exhibited a lower value on the cytokine panel during the course of healing, although depending upon a therapeutic drug, and did not act in contact with the clinical condition (see FIG. 5). However, at the time of remission, the value of IL-4 was sequentially normalized, and hygiene hypothesis (Th1/Th2 ratio) was also improved. Different from Th9 (IL-9), Th17 (IL-17) and Th22 (IL-22) were not effective for the parameter at the time of healing severest or severe atopy. In an improper order for bio-parameter, specific IgE, total IgE amount, TARC and TGF-β1 could be arranged.

The bar chart of FIG. 5 shows the scores, in the order from left, IL-1ra (IL-1 receptor antagonist), IL-2, IL-4, IL-5, IL-6, IL-7, IL-8, IL-9, IL-10 with time elapse between pretreatment (day 0) of the severest atopy (erythema and pruritus over 36% of the whole of the skin) and Day 74 after treatment.

### Example 8

### Administration Effect of Zinc Finger Znf/PAD Upon Severest Atopic Dermatitis

A sample was male in late 50s having 49 years morbidity. At the time of the first medical examination (at the time of starting the treatment), and he was be diagnosed presumptively to be severest atopic dermatitis that 36% or more of the whole skin was skin erythema. Hyperimmunolobulinemia was also observed. The treatment was performed by oral administration of “zinc finer Znf/PAD” (also simply referred to as zinc finger Znf) (3.1 mg/kg as real amount of zinc yeast or 2 g as an upper limit).

The judgment of the therapeutic effect was made according to the cytokine panel test or the evaluation standard described herein (found by the present invention). The results are shown in Table 11.

TABLE 11
Clinical Effects of Atopy Patient at Time of Novel Zinc Finger upon
Cytokine Panel

Zinc Finger Znf*

| | Before | After |
|---|---|---|
| | Treatment | Treatment |
| Zinc concentration in Serum (ng/ml) | 63 | 87 |
| Th1/Th3 Vital Reaction (%) | 5.7/0.2 | 12.5/6.2 |
| TARC Th2 Chemokine (ng/ml) >700 | 21.800 | 690 |
| IL9 Th9 (pg/ml) | 10.4 | 2.9 |
| IL13 Znf Activity (pg/ml) | 5.9 | 25.6 |
| FGF Znt + Znf Activity (pg/ml) | 23.2 | 63.5 |

As can be seen in Table 11 (clinical effect of atopy patient at the time of administration of zinc finger Znf/PAD), the patient has characteristics of severest atopic dermatitis, in which Th1 and Th3 vital reactions were low values, which is characteristic of the severest atopic dermatitis, and TGF-β was as high as 27 pg/ml. Th2 vital reaction is decreased by the treatment. IL-4, which is Th2 cytokine, had a low relation with therapeutic effect, but IL-9 cell mainly produced from Th9 cell (discovered as atopy allergy cell: also confirmed to exist in the dog) had a correlation with the therapeutic effect and was suppressed by the administration of zinc finger Znf/PAD. Furthermore, IL-13 produced by the activation of Znf having DNA repair capacity via zinc signal transmission was significantly increased (Table 11).

While IL-4 and IL-13 exist in the signal transmission system and promote the production of antibody such as IgE, in the course of treatment of atopic dermatitis there was no relation between IL-4 and IL-13, and IgE was significantly suppressed (data not shown). The relation between treatment course and IgE in the increasing of production of IL-13 was negative, the production of IL-13 was involved in the treatment of the sample via the activation of Znt and Znf. Furthermore, Znt and Znf involved the hyperplasia of connective tissue such as bones and skins and from the viewpoint that the skin condition was improved, the production of IL-13 has been indicated to have a relation with DNA repair.

In the course of the treatment described above, the skin of hand (back of the hand) is thick, rough feel (lichenification), scratching were marked before the treatment. After the treatment, the skin was substantially back to normal, became smooth, and scratching was disappeared (not shown). The depigmentation of scratching mark could be seen.

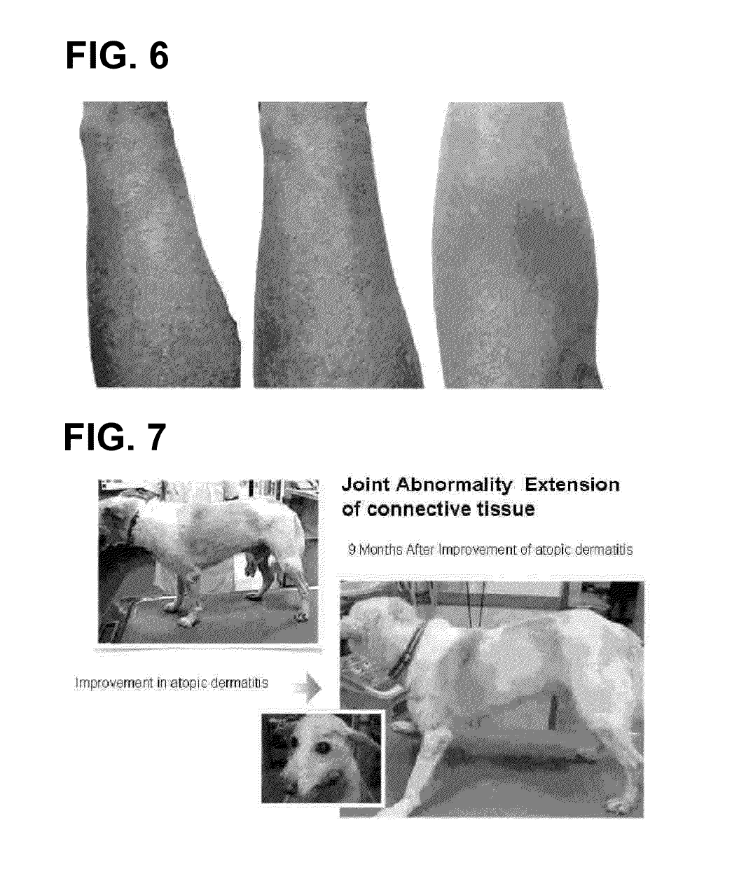

In the case of the front arm (FIG. 6; from left side pretreatment, under the treatment, 232 days after treatment), while the skin was thick, rough and sticky feel (lichenification) as in the hand (Left side figure of FIG. 6), rough and sticky feel were disappeared and smoothness could be seen and the red of the skin (dermatitis and inflammation) was disappeared.

### Example 9

### Administration Effect of Zinc Finger Znf/PDA Upon Ehlers-Danlos Syndrome (EDS) After Treatment of Dog Th2 Atopy

### (Case)

### Kind: Mix

### Sex: Male

Age: 11 years and 3 months old

The sample exhibits EDS from Th2 atopic dermatitis by Th3 march (see FIG. 7).

After atopic dermatitis had been confirmed by Th examination and others, the sample was medically treated to remit the atopy (FIG. 7 Left side). After nine months, the head and mantle were observed. The dehairing in a map form at an upper portion of left eye, typical scapular region descent, and opening foreleg were observed (FIG. 7 right).

Appearance of Symptom: As typical clinical symptom of 5 items, (3) excess extension of leg joint and (5) excess extension of skin and vulnerability of skin (lower Th1 and Th2 values) were observed.

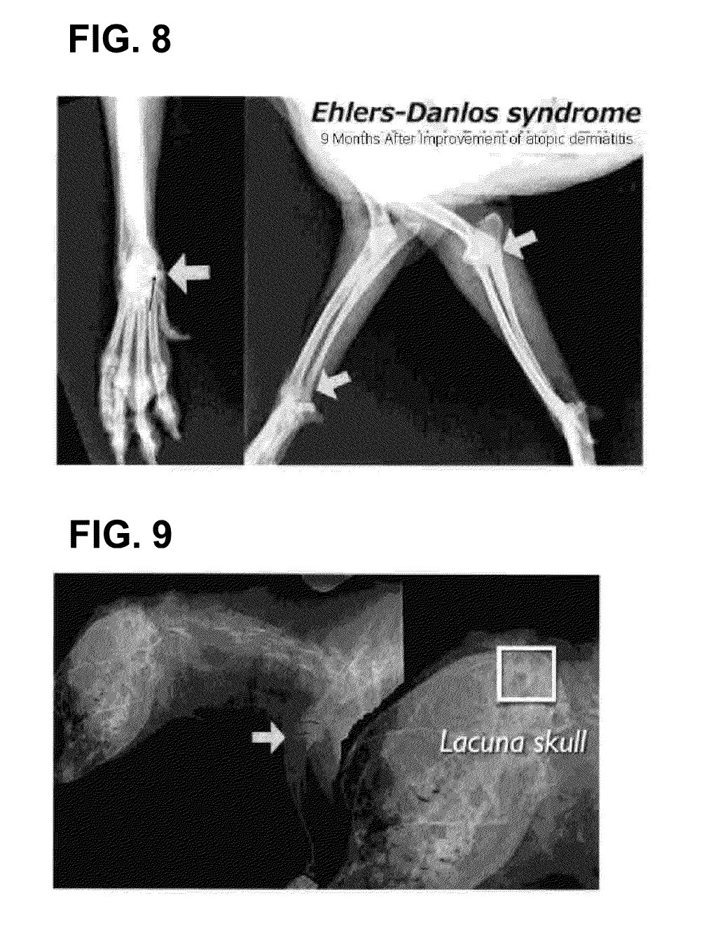

When we suspected EDS which was not seen at the time of the treatment of atopy, and when plain x-ray examination was conducted (FIG. 8), although no abnormality was seen, multiple dislocations were observed at a joint easy to be loaded (FIG. 8 arrows). According to the cytokine panel test, EGF was significantly low value which was 0.5 μg/ml. From the cytokine panel test, it was determined to be dog EDS.

The treatment was conducted by oral administration of zinc finger Znf/PAD (3.1 mg/kg as real amount of zinc yeast). By the administration of zinc finger Znf/PAD, FGE and Znf activity (IL-13) were increased in comparison with before treatment and one month after the treatment, dehairing at the portion above the eyes was improved.

### Example 10

### Effect of Administration of Zinc Finger Znf/PDA Upon EDS Via Th3 March as Dog's Hip Dislocation and Cardiomegaly

### (Case)

### Kind: American Cocker Spaniel

Sex: Female (contraceptive Treated)
Age: 2 Years and 11 months old
Th3 March: As the appearance of the symptom, first hip dislocation appeared and improved about 4 months. Thereafter, heart disease concurred, and heart murmur disappeared along with the treatment of EDS. The subject marched Paget's disease of bone (see radiographic finding of FIG. 9).

As typical clinical symptom of 5 items, (3) excess extension of leg joint, (4) excess extension of knee joint, and (5) excess extension of skin and vulnerability of skin (lower Th1 and Th3 values) were observed.

The evaluation of the therapeutic effect was conducted by the zinc concentration in blood serum, plain x-ray examination, the cytokine panel test, and the evaluation standard described herein (found in the present invention).

In FIG. 9, bone resorption (lacuna skull) was observed. There coexisted bone resorption and bone augmentation in this case, bone becoming poor and bone resorption were mainly observed. Bone Behcet's easily induces bone cancer (human).

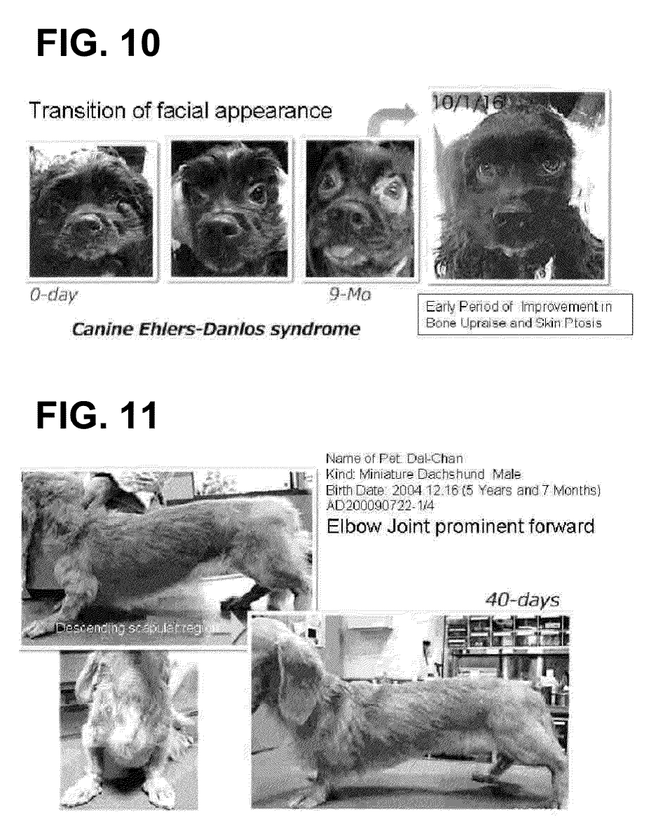

The treatment was conducted by oral administration of zinc finger Znf/PAD (3.1 mg/kg as real amount of zinc yeast). The zinc concentration in blood serum before treatment was 42 ng/ml, and by the administration of zinc finger Znf/PAD, the zinc concentration was recovered to be 50 ng/ml. According to the cytokine panel test, FGF was low value (separately see examples of cytokine panel). Findings of excess extension of skin (FIG. 10, 0 Day), protrusion of compound skull fracture due to hyperplasia; revealment of tongue and dehairing around eyes thereafter (FIG. 10 9Mo); and findings after 9 months (9-Mo) were shown. Because of young, coexistence and duplicated Th3 march diseases were observed. The brittleness of connective tissue and excess extension reached their peaks at nine months. By the administration of Znf/PAD, the heart disease was improved and the conditions of skin were significantly improved (most right side figure). It was also considered to be clinical transition by BMP and TGF-β1 signaling.

### Example 11

### Effect of Administration of Zinc Finger Znf/PDA Upon EDS Via Dog's Th3 March

### (Case)

### Kind: Miniature Dachshund

### Sex: Male

Age: 5 Years and 7 months old

As typical clinical manifestation amongst 5 items, all items (1) digitus primus was dorsiflexed to attach forearm, (2) digitus secundus to digitus quintus were dorsiflexed to be bent 90 degree or more, (3) excess extension of leg joint, (4) excess extension of knee joint, and (5) excess extension of skin and vulnerability of skin (lower Th1 and Th3 values) were observed, and in addition, vulnerability of the skin (lower Th1 and Th3 values) were observed.

The treatment was performed by oral administration of “zinc finer Znf/PAD” (3.1 mg/kg as real amount of zinc yeast or 2 g as an upper limit). The results are shown in FIG. 11. By 40 days' administration of zinc finger Znf/PAD, connective tissue was significantly improved to normalize the ptosis of scapular region. All of clinical scores of EDS were improved (FIG. 11).

### Example 12

### Early Intervention in Th3 March

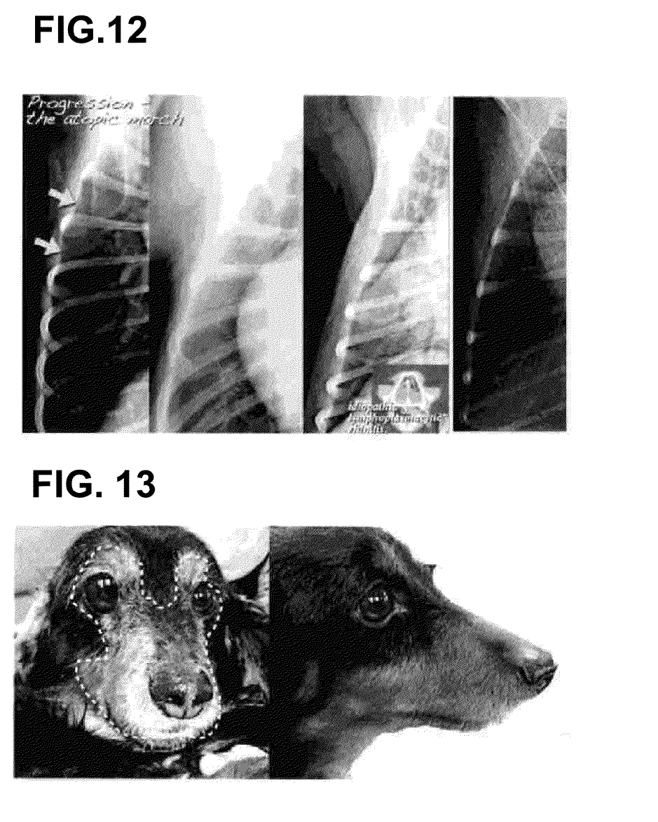

FIG. 12 shows plain x-ray examination and CT image (small cut image) of a dog's severest atopic dermatitis (including evaluation of Th examination) and asthma (chronic tracheitis and eosinophilic pneumonia) marched thereafter. FIG. 12 shows an example of evaluating intervention of early medical treatment (early intervention of diseases) in march pneumonia (early intervention by a method for selecting a drug by Th3 march and examination).

In FIG. 12, in the course of Th3 march according to the age, the subjects exhibited asthma (atopy, chronic bronchopneumonia, asthma, pollinosis/idiopathic lymphocytic plasmic rhinitis CT) and FIG. 12 shows the ingravescence course (3 figures from light side), and findings of plain x-ray examination after the early intervention at the time of healing (most right side of FIG. 12). In the drawing showing the ingravescence course, the findings of progressed, severe, right side pulmonary lobe (third cut drawing from left) were described together. The arrow portion shows findings of miniaturizing lung field at the eosinophilic infiltration. By the administration of “zinc finger Znf/PAD”, the value of IL-13 (index of activation of Znf in Item I of the cytokine panel test) after the administration was increased

### Example 13

### Shifting of Th2 and Th3 Vital Reactions and Th3 March and Chronic bronchopneumonia/Asthma

### (Case)

### Kind: Miniature Dachshund

### Sex: Male

### Age: 9 Years old

It can be confirmed that atopic dermatitis could be treated by the standard treatment to withdraw the drug after 39 days (FIG. 13, left side figure: pretreatment, right side figure: post-treatment)

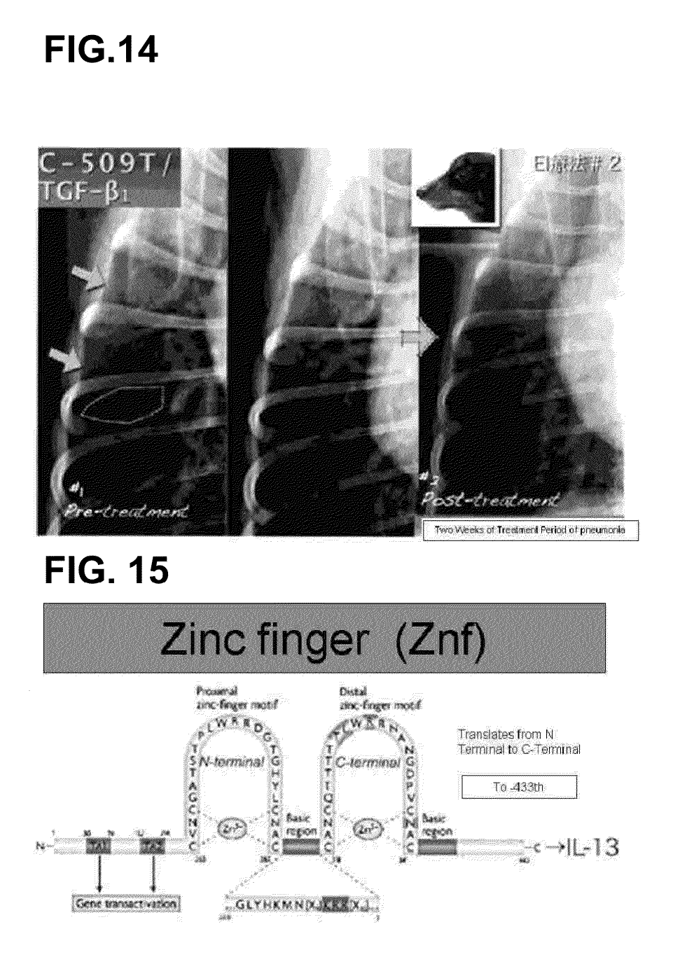

At the time of first medical examination (initiation of treatment of atopic dermatitis), the measured value of active TGF-β1 was 16.8 ng/ml, which was higher than 19 and the subject had anamnesis of mild cough; thus plain x-ray examination was conducted considering Th3 march. The findings at this time are shown in FIG. 14 (Left side figure: Pretreatment, Middle side figure: Course of Treatment, Right side figure: Post-Treatment). Vasculitis and miniaturization of lung field were seen in bronchus around the trachea and at periphery of lung field, indicating march chronic bronchopneumonia/asthma. Particularly, the cervical trachea was collapsed (continuance of remodeling of trachea cartilage, delaying: ballooning), and meandering accompanying with remodeling trachea was visualized. At the same time, strong consolidations were come across at right and left medial lobes, and localization of inflammation of the lung field could be seen (FIG. 14 Left side figure, Arrowed portion).

In order to suppress remodeling of trachea cartilage, the value of TGF was regulated and “zinc finger Znf/PDA” was administrated as the therapeutic drug to apply early intervention. As a result, abnormality in the respiratory system was developed behind in skin disease, and consequently, the drug could be withdrawn at a time earlier than the period of treating atopy. In the intervention of early medical treatment (early intervention of diseases), the disease developed after march has been indicated to have no untreated period. This evaluation was conducted by Th vital reaction test or the cytokine panel test.

### Example 14

### Effect of Administration of Zinc Finger Znf/PAD Upon Improvement of Mental Developmental Disorder in Th3 March and Atopy

It has been clarified that the severest atopic dermatitis is the diseases, which can make a treatment plan by the cytokine panel test at the downstream of zinc cell cytokine signal transmission/Th march. Particularly, it has also been clarified that in the treatment of the individual with the severest atopic dermatitis, this disease is a skin disease which cannot be cured without Th vital reaction test and the cytokine panel test. While TGF-β1 (Th3 cytokine) and Th3 vital reaction are signal transmission systems, when the abnormality of Znt exists, although TGF-β1 is produced, Th3 vital reaction is decreased. In this case the relation (dissociation), in which the value of plasma active TGF-β1 is high, the measured value of Th3 vital reaction (particularly endogenous reaction) is low (not more than 1.32%), can be observed.

In the patient of severest atopic dermatitis, and also in the patient in 20s of middle atopic dermatitis whose Th1 vital reaction and Th3 vital reaction were decreased, low level (not more than 1.32%) of plasma active TGF-β1 and Th3 vital reaction (particularly endogenous) were observed. From these cases, mental development disorder (developed by inactivation of Znf/Znt) and decreasing of IL-13, in a zinc concentration in the serum not more than 55 ng/ml) can be seen. When the zinc concentration in the serum is increased 10 ng/ml to be recovered to not less than 65 ng/ml, autonomic nerve tone abnormality, depressive psychiatric symptom, decreasing of ADL were improved. In particular, mental development disorder due to decreasing of zinc concentration in serum was significantly found in the elderly, and in Th1 dependent skin disease 60% of depressive psychiatric symptom could be seen. By the administration of “zinc finger Znf/PAD”, the depressive psychiatric symptom due to decreasing of Znt (zinc transporter) or decreasing of zinc concentration in serum (not more than 52) was improved through the increasing of IL-13.

### Example 15

### Outline of Example

We have discovered that there is Th3 march seen in subsequent to Th2 atopy march (Atopy March, Th2 March) before report for typical Th2 atopy march (Suzuki Y, Kodama M, Skin barrier-related molecules and pathophysiology of asthma., Allergol Int., 2011 March; 60(1):11-5.; Spergel J M., From atopic dermatitis to asthma: the atopic march., Ann Allergy Asthma Immunol., 2010 August; 105(2): 99-106.). In conventional, there is a report that zinc finger (Znf) nucleases were artificially inserted in a nucleus (Tomoji Mashimo et al., Generation of Knockout Rats with X-Linked Severe Combined Immunodeficiency (X-SCID) Using Zinc-Finger Nucleases., PLoS One., 2010 Jan. 25; 5(1):e8870), we have discovered “zinc finger/Znf” (see Table 5 above), and indicated clinical effects by oral treatment.

However, there is no report that responsible genes for dog or human atopy (Barros Roque J., Haplotype sharing excludes canine orthologous Filaggrin locus in atopy in West Highland White Terriers., Anim Genet., 2009 October; 40(5):793-4.; Cai S C et al., Filaggrin Mutations are Associated with Recurrent Skin Infection in Singaporean Chinese Patients with Atopic Dermatitis., Br J Dermatol. 2011 Jul. 25.) induces Th3 march, and also there is no report that mammal Ehlers-Danlos syndrome (EDS) is included in Th3 march. Even more, there is no report that EDS is improved by zinc finger/Znf and atopy march related genes can be repaired by the oral administration.

In this example, it has been clarified for the first time that Th3 march (Th1, Th2, and Th3 diseases) is due to deletion or mutation of filaggrin gene (atopy responsible gene). In addition, it has been indicated for the first time that these march diseases (Th1 diseases such as vulgaris plaque psoriasis, Th2 diseases such as atopic dermatitis, and Th3 diseases such as contact dermatitis, human or dog EDS) can be treated or improved by the oral administration of zinc finger/Znf to thereby repair deleted or mutated DNA of filaggrin gene also relating to Th3 march (including induction of FGF and IL-13).

### <Experimental Methods and Materials>

### (1) ELISA Kit for Measuring Filaggrin (FLG)

A kit for sandwich enzyme immunoassay was used for quantitatively in vitro measuring filaggrin (FLG) proteins (gene related filaggrin) in a tissue homogenate or other body fluids.

Specifically, as for detection strength, measurement was done at a wavelength of 450 nm. Biotin-coupled polyclonal antibody specifically coupled with FLG was used, and the actual measured value of filaggrin (ng/ml frozen serum) was roughly estimated by a standard curve (FIG. 17) previously prepared with FLG standard sample.

### (2) Calculation of Filaggrin Repair Capacity by Zinc Finger/Znf

Amongst mammal patients (human and dog etc.) having Th3 march/test animals, patients an amount of filaggrin proteins based on filaggrin (FLG) gene which is responsible gene of atopy syndrome (atopy is newly defined as atopy syndrome by the finding of filaggrin) were not detected or were detected in a small amount (deletion or mutation of filaggrin gene) were selected (Th3-FLG).

Subsequently, “zinc finger/Znf” (see Table 5 above) was administrated to the patients etc. (Th3-FLG) in which deletion etc. of filaggrin were confirmed, after which filaggrin repair activity was compared. The term “filaggrin repair activity” used herein is assumed as an enhancement rate of the amount of filaggrin protein produced, which can be considered to be repair capacity of filaggrin DNA (repair rate).

The filaggrin repair capacity (% of control) by zinc finger/Znf was calculated according to the following formula:

Filaggrin repair capacity (% of control)=[(Amount of FLG (ng/ml) after administration of zinc finger/Znf)/(Amount of FLG (ng/ml) at the time of no administrating zinc finger/Znf)]*100%

### (3) Administration Method and Administration Period of Zinc Finger/Znf

Preferable condition of administrating amount was “zinc finger/Znf capsule/head/BIO PO. A preferable administration period was from 60 days to 120 days. Intake can be made by an administration method such as intraoral application.

### Example 15-1

### Repair of Atopy Responsible Gene in Th3 March

The repair capacity of FLG and the clinical effect upon the patient of Th3-FLG having the following profile by zinc finger/Znf were confirmed.

### Outline of Patient (Profile)

Female (Domestic Help) 26 Years Old, Th3-FLG Patient

She developed atopy when she was kindergarten child, and no steroid has been used at all.

She was low IgE atopy patient, and had pollinosis amongst complete Th2 march.

Her atopy morbidity history is 19 years.

### Filaggrin Repair Capacity:

Over a period of 103 days, zinc finger/Znf was administrated to the patient.

The FLG repair capacity by zinc finger/Znf was 116.7%, and DNA repair was observed.

### Clinical Effects:

The atopy morbidity period is 19 years but her erythroderma was relatively mild. The patient was concurred immunosuppressive fibrositis due to abnormality of zinc cell signal.

As described above, the administration period of zinc finger/Znf was 103 days, and FLG repair was approximately 1.2 times (120%). By the administration of zinc finger/Znf, erythroderma was completely improved as shown in FIG. 18 (in this figure, −Zn indicates pretreatment and +Znf indicates post-treatment). Although Th1 vital reaction remained decreased, the therapeutic drug could be withdrawn.

### Example 15-2

The repair capacity of FLG and the clinical effect upon the patient of Th3-FLG having the following profile by zinc finger/Znf were confirmed.

### Outline of Patient (Profile)

Female (housewife) 49 Years Old, Th3-FLG Patient

She developed atopy on several months olds, a large rash came out on a head. From kindergarten, itch was in a normality state. An ointment or steroid ointment was used to control pruritus. At the time of high school, skin conditions were changed for the worse and, the steroid ointment was applied onto the face. After graduating from high school, the use of steroid was withdrawn. After the marriage, a steroid was used at one time. At high school, she had asthma and recovered by steroid, and then she developed pollenosis (atopy march due to deletion of FLG). Due to anaphylaxis, she had taken to hospital by ambulance. Her skin was in a grater-like state, the collection of blood from her arm was difficult.

She was a low IgE atopy patient, and had Th3 march containing 6 diseases of Th2 march. She also had latex fruit syndrome. She had oldest daughter having atopy. Her parent had atopic dermatitis.

Her atopy morbidity history is 49 years.

### Filaggrin Repair Capacity:

Over a period of 103 days, zinc finger/Znf was administrated to the patient.

The FLG repair capacity by zinc finger/Znf was 200% (twice), and DNA repair was observed.

### Clinical Effects:

Similar to the atopy patient 26 years old, the patient was concurred immunosuppressive fibrositis due to abnormality of zinc cell signal and had a health history of atopy march diseases seen by FLA deletion (6 diseases such as asthma). In addition, she had a health history of anaphylaxis, and was complete Th3 march. As skin lesion, seborrheic dermatitis, rubbing marks, hand eczema accompanying with “grater like skin” were significant. Also, she had complicated fruit syndrome.

As described above, the administration period of zinc finger/Znf was 103 days, and FLG repair was twice (200%). The improvement effect of hand eczema by the administration of zinc finger/Znf is shown in FIG. 19 (in this figure, −Zn indicates pretreatment and +Znf indicates post-treatment). Also, as for a symptom due to deletion of FLG, the effect of the heavy hand eczema is specifically shown in the following table.

| | −Znf | +Znf |
|---|---|---|
| Scratch mark | +++ | ++ |
| Roughness of Skin | +++ | + |
| Skin Flexibility | − | + |
| Atopy Invasion | +++ | + |
| Mobility of thumb* | + | − |
| Mobility of little finger* | + | − |

*Hand working and wet work such as cooking could not performed.

### Example 15-3

The repair capacity of FLG and the clinical effect upon the patient of Th3-FLG having the following profile by zinc finger/Znf were confirmed.

This example is an example of treatment of Th2 march patient from Th1 disease (psoriasis).

In psoriasis, type I cytokine such as IFN− is produced in a large amount, and as for infiltrated Th cell, Th1 is predominant. On the other hand, in skin rash portion of psoriasis, in addition to Th1, various types of Th cells such as Th17 and Th22 cells are infiltrated. In the case of dog, the diseases march from Th1 to Th2. Due to the increasing of FLG, it has been clarified that it is in the course of Th3 march.

### Outline of Patient (Profile)

Male: 52 Years Old, Th-FLG Patient (Th1 Disease Vulgaris Psoriasis/Th3 March Disease)

Disease is vulgaris psoriasis, which is Th1 disease.

His morbidity history is 26 years.

### FLA Repair Capacity:

(1) For referential example (control experiment), zinc agent (Trade name: Promac) was tried to be administrated to this patient. Since the activated TGF-β1 was as high as 9.3 ng/ml (average value of healthy subjects: 1.1 ng/ml), a novel supplement TgF was administrated to regulate TGF-β1. Thereafter, Promac was orally administrated to the adult twice per day in an amount of 75 mg per one dosage, or 1.6 mg/kg at a time after breakfast and a time before bedtime as polaprezinc. Promac was administrated for 61 days. As a result, FLA repair by Promac could not be recognized and no clinical effect could be recognized.
(2) On the other hand, zinc finger/Znf was administrated to the patient for 74 days.

The filaggrin repair capacity by zinc finger/Znf was 751.3% (7.5 times) and DNA repair was observed

### Clinical Effects:

As described above, administration of zinc agent (control experiment) had no effect. At this time, filaggrin was 0.9 ng/ml and filaggrin abnormality was confirmed and, thus, administration of zinc finger Zn/f was conducted. The increasing of FLA by zinc finger/Znf approached 7.5 times. From this fact it has been revealed that Th1 disease has a series of pathology induced from filaggrin, which is an atopy responsible gene, which is Th2 disease, i.e., Th3 march.

This case (vulgaris psoriasis) is a disease presenting hyperkeratosis and is characterized by scale. As shown in FIG. 20, in the state shown in light figure before administration of zinc finger/Znf (−Znf), the scales were fallen as much as they could be visualized only for one day. In the state shown in right figure after administration of zinc finger/Znf (+Znf), the scales were stored on the floor and could not be observed. The skin pathology was also improved.

### Example 15-4

In this example, to patients or test animals (Th3 EDS-FLG), who were affected with atopy at a young age, and were confirmed to be Ehlers-Danlos syndrome (EDS) by skin extension, zinc finger/Znf was administrated to confirm the clinical effects thereof.

### Selection of Patients:

Amongst Th3 march patients, mammals (human and dog) having Ehlers-Danlos syndrome (EDS) (patients having atopy syndrome at first and then Ehlers-Danlos syndrome) (Th3 EDS-FLG) were selected.

Decision to be Th3 EDS-FLG was conducted by the presence or absence of skin extension (loose skin being excluded out) after affection with atopy syndrome.

### Measurement of EDS Skin Extension Rate:

EDS skin extension rate (% of Skin-extension) is calculated based on the following formula. It was provisionally diagnosed to be hyperextension at 14.5% or more in dog, 19.5% or more in cat, and 1.0% or more in human (at least one joint of the finger joint, or according to the criterion of EDS in each case). New EDS was selected by varix, eye disease, deformation of joint, partial anodontia, and bone dysgenesis. In the selection, the targets were patients having underlying disease of Th2 march.

EDS Skin Extension Rate (% of Skin Extension)=[Skin Extension at blade bone region (cm)/body height or length of body (cm)]*100.

### Confirmation of Decreasing Effect of EDS Skin Extension Rare by Zinc Finger/Znf

In both cases of human and dog, the patients falling under criterion of respective syndromes of EDS including skin extension while possessing atopy morbidity history (at least 2 years in animals, and at least 10 years inhuman) were selected.

To the patients etc. (Th3 EDS-FLG), who were affected with atopy at a young age, and were confirmed to be Ehlers-Danlos syndrome (EDS) by skin extension, zinc finger was administrated and the skin extension rates % before and after administration were compared.

### Example 15-4-1

Upon dogs (five cases) judged to be Th3 EDS-FLG having the following profiles, the clinical effects by the administration of zinc finger/Znf were confirmed.

### Outline of Dog Patients (Profile)

To 5 cases of dog Th3 EDS-FLG affected with atopy and confirmed to be EDS by skin extension rate, zinc finger/Znf was administrated (Pre-Znf) to evaluate the suppression effect upon skin extension. In the case where IL-4 in serum or Th2 vital reaction was high, no suppression of skin extension could be observed.

### Clinical Effects:

The decreasing effects of skin extension rate upon dog Th3 march EDS-FLG by zinc finger/Znf were confirmed.

As for the 5 cases having high skin extension rate, when the effect after the administration of zinc finger/Znf for 40 days were confirmed, statistical differences (P<0.05) could be seen in all cases as shown in FIG. 21 (Pre-Znf is at the time of administration, Post-Znf is 40 days after the administration). Particularly, in two cases, 40 days after the administration, dog EDS skin extension rates were under the 14.5, which is threshold value of dog EDS skin extension rate, and their skins were returned to normal skins.

### Example 15-4-2

Upon dogs judged to be dog Th3 march EDS-FLG, clinical effects at the time of normalization of skin extension rate by the administration of zinc finger/Znf were confirmed.

### Outline of Dog Patients (Profile):

### Dog (American Cocker Spaniel) Female, 4 Years and 2 Months Old

From young dog, ulnar drift of foreleg, subluxation of hip joint and the like could be seen, and atopic dermatitis was developed at one year old. Thereafter, a treatment of withdrawing steroid was conducted and serious treatment was conducted from 1 and half years before, skin lesion was repeatedly relapsed. The skin lesion had well-creased dry scaly skin considered to be FLG deletion. Approximately 200 days after first medical examination, hyperextension of the skin was suspected, and as a result of measurement of skin extension rate (16.5%), the patient was provisionally diagnosed to be EDS (decided to be Th3 march case).

### Clinical Effects:

The clinical effects by the administration of zinc finger/Znf were confirmed at the time of normalization of the skin extension rate.

Zinc finger/Znf was administrated for 69 days. Findings of cervical region at the abdomen side before administration (−Znf) and 69 days after administration (+Znf) are shown in FIG. 22. In this figure, an upper portion is a head side and a lower portion is a tail side.

In this case, in the course of the medical treatment of atopic, loose skin at the cervical region at the abdomen side (sagging) and alopecia were seen (Left side figure of FIG. 22: −Znf). In general, similar to the human case, the dog EDS involves hyperlinearity seen in FLG deletion and skin hypertension in which lesion cervical region of head and rear body are easily moved. In this case, all characteristic skin symptoms were observed. Since symptoms of convergence and relapse were repeated gradually, when the skin extension rate was measured, it was over 14.5 which is the standard of EDS. The administration of zinc finger/Znf 1 capsule/head/BIO PO was initiated.

By the administration of zinc finger/Znf, the skin extension rate was 0.61% improved (effect of decreasing hypertension), and 359 days after treatment, the rate was under 14.5, which is threshold value of dog EDS skin extension rate, and reached 9%, the skin hypertension was normalized although hair loss remained observed.

In the past, EDS is assumed as disease without a cure having no treatment method, the treatment effects by the administration of zinc finger/Znf can be recognized for the first time.

It has been considered that zinc finger has low improvement effect for directly improving symptom, but DNA repair capacity has been clinically confirmed by us and thus, zinc finger/Znf can be considered to have an improvement effect.

### Example 15-4-3

The subject was a patient having the following profile who had taken to hospital by ambulance due to anaphylaxis shock to be hovering between life and death. Particularly, as for the patient having a clinical course of mucosa lesion such as skin and gum, ophthalmologic diseases (astigmatism and retinal detachment), and partial anodontia over three or more years after anaphylaxis shock, the early intervention by zinc finger/Znf was conducted.

### Outline of Patient (Profile):

### Male, 61 Years Old, Human Th3 March-FLG

At a time of junior high school, it was pointed out from a school doctor to be astigmatism. When he was a high school student, he had auditory clogged cooped-up feeling (symptom continued for 5-6 hours when developed). When he was about 40 years old, he had an anamnesis of duodenal ulcer. He was affected with atopic dermatitis including seborrheic dermatitis, and due to anaphylaxis shock at the last stage of Th2 march, he had taken to hospital by ambulance to produce loss of consciousness (at Jun. 14, 2007). At the time, dry mouth and tooth ache were recognized, due to the pain, intake of solid was difficult. The tooth ache (gingivitis) was continuously observed over a period of 5 years. Partial anodontia which is a symptom of new dysostosis EDS, was pointed out. There were pathogenic histories of skin eczema (atopy march) in third degree of kinship.

This patient was confirmed to have filaggrin deletion. The patient was decided to be atopy march by the deletion of filaggrin gene, and to be Th3 march by Th test etc.

### Clinical Effects:

Zinc finger/Znf was totally administrated for 63 days.

First, a step treatment including zinc finger/Znf was applied for 30 days. FIG. 23 shows a dental panorama image before and after treatment. About 10 days after administration he could take a solid, the toothache (gingivitis) was gradually moderated and then disappeared. According to dental panorama radiographic findings after improvement of clinical symptom, floating teeth (45^{th} day: vertical bone resorption) was changed for the better (Cut figure of lower left figure of FIG. 23).

### INDUSTRIAL APPLICABILITY

According to the present invention, a method for treatment and prevention, particularly early intervention of Th3 march-related diseases including Th2 march by filaggrin deficient gene (including mutant), and specifically a pharmaceutical composition which can previously prevent or treat in an early time a disease supposed to be developed in the next stage due to the marching can be provided. Also, a method for treating, preventing, diagnosing the disease concerned, a method for testing classification of treatment and prevention, and a method for detecting intention to use a therapeutic drug and a prophylactic and kits for using the same utilizing a novel zinc finger (Z-finger), which can realizes previous prevention of Th3 march-related diseases, can be provided.

1. A pharmaceutical composition for treatment and/or prevention of Th3 march-related diseases comprising zinc, calcium and phosphor.

2. The pharmaceutical composition according to claim 1, which further comprises pumpkin seeds and corn silk.

3. The pharmaceutical composition according to claim 1, which activates DNA repair capacity.

4. The pharmaceutical composition according to claim 1, which activates DNA repair capacity of zinc finger.

5. The pharmaceutical composition according to claim 1, which activates repair capacity of deletion or mutation of filaggrin gene DNA.

6. The pharmaceutical composition according to claim 1, wherein Th3 march-related disease is selected from at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3.

7. A method for treating and/or preventing Th3 march-related diseases comprises:
measuring a concentration of cytokine and/or chemokine in blood of a test animal; and
initiating, continuing, interrupting or discontinuing an administration of the pharmaceutical composition according to claim 1 using the measured results as an index.

8. A method for examining the classification of treatment and/or prevention of Th3 march-related diseases comprising:
measuring a concentration of cytokine and/or chemokine in blood of a test animal; and
examining the classification of treatment and/or prevention of Th3 march-related diseases using the measured results as an index.

9. A method for deciding intension to use of treatment and/or prevention drug comprising:
measuring a concentration of cytokine and/or chemokine in blood of a test animal; and
deciding intension to use of treatment and/or prevention drug using the measured results as an index.

10. A method for diagnosing Th3 march-related diseases comprising
measuring a concentration of cytokine and/or chemokine in blood of a test animal; and
diagnosing Th3 march-related diseases using the measured results as an index.

11. The method according to claim 7, wherein said cytokine and chemokine is at least one member selected from TGF-β1, IL-1β, IL-4, IL-5, IL-6, IL-9, IL-10, IL-13, IFN-γ, RANTES, various FGF (Healing Formula).

12. The method according to claim 7, which further comprising measuring a concentration of zinc in blood.

13. The method according to claim 7, wherein Th3 march-related disease is selected from at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3.

14. The method according to claim 7, wherein said test animal is a human or non-human animal.

15. The method according to claim 13, wherein non-human animal is a dog.

16. A kit for examining the classification of treatment and/or prevention of Th3 march-related diseases comprising an antibody against a cytokine and/or chemokine.

17. A kit for deciding intension to use of treatment and/or prevention drug of Th3 march-related diseases comprising an antibody against a cytokine and/or chemokine.

18. A kit for diagnosing Th3 march-related diseases comprising an antibody against a cytokine and/or chemokine.

19. A kit for treatment and/or prevention of Th3 march-related diseases comprising an antibody against a cytokine and/or chemokine and The pharmaceutical composition according to claim 1.

20. The kit according to claim 16, wherein said antibody is arrayed on a bead.

21. The kit according to claim 16, wherein Th3 march-related disease is selected from at least one disease selected from Th1 related plaque psoriasis, Th2 related atopic dermatitis, and Ehlers-Danlos syndrome, Paget's disease of bone, enteropathic acrodermatitis syndrome, external otitis, gastritis, enteritis, asthma, pyoderma, hypersensitivity pneumonitis, chronic tracheitis, pollinosis, allergic rhinitis and anaphylaxis relating to Th3.

22. The kit according to claim 16, wherein said cytokine and chemokine is at least one member selected from TGF-β1, IL-1β, IL-4, IL-5, IL-6, IL-9, IL-10, IL-13, IFN-γ, RANTES, various FGF (Healing Formula).
